(19) United States
(12) Patent Application Publication
(10) Pub. No.: US 2024/0342168 A1
(43) Pub. Date: Oct. 17, 2024

(54) SMALL MOLECULE INHIBITORS FOR ANTI-CANCER COMBINATION THERAPY

(71) Applicant: UNIVERSITY OF CAPE TOWN, Rondebosch, Cape Town (ZA)

(72) Inventors: Virna Drucille LEANER, Rondebosch (ZA); Pauline Janet VAN DER WATT, Rondebosch (ZA); Ru-pin Alicia CHI, Rondebosch (ZA)

(21) Appl. No.: 18/294,596

(22) PCT Filed: Aug. 2, 2021
(86) PCT No.: PCT/IB2021/057054

§ 371 Date: Feb. 2, 2024

(51) Int. Cl. A61K 31/4985; A61K 31/5377; A61K 33/243; A61P 35/00

(52) U.S. Cl. CPC A61K 31/4985; A61K 31/5377; A61K 33/243; A61P 35/00

(57) ABSTRACT

A combination for use in treating cancer is provided. The combination comprises a therapeutically effective amount of a quinoxaline derivative of Formula I which inhibits nuclear import protein, Kpnβ1, and a therapeutically effective amount of at least one platinum-based chemotherapeutic agent. The combination therapy provides an enhanced anti-cancer therapeutic effect compared to the effect of the compound of Formula I and the at least one platinum-based chemotherapeutic agent administered alone. Formula I

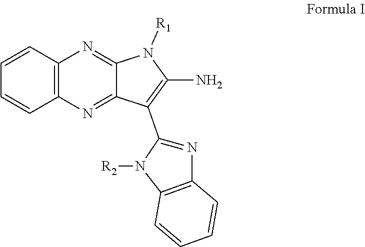

### FIELD OF THE INVENTION

This invention relates to small molecule inhibitors for anti-cancer combination therapy. In particular, it relates to the use of small molecules that inhibit protein transport into the nucleus of cells to enhance the sensitivity of cancer cells to chemotherapeutic agents and obtain an enhanced anti-cancer effect.

### BACKGROUND TO THE INVENTION

Karyopherin beta (β) 1 (Kpnβ1) is a nuclear transport protein involved in the import of cargo proteins and RNAs across the nuclear pore complex, from the cytoplasm into the nucleus. Kpnβ1 is the major importing machinery in mammalian cells and traffics cargoes from the cytoplasm into the nucleus in interphase cells. Through shuttling a wide range of cargoes, Kpnβ1 regulates numerous cellular functions including inflammation, migration, apoptosis, morphology, circadian clock function and others. In addition to its nuclear importing function in interphase cells, Kpnβ1 also mediates cell division by regulating spindle assembly and mitotic exit, thereby exhibiting pleiotropic functions in maintaining cell homeostasis and division.

Owing to its deregulation in multiple cancers, Kpnβ1 has been studied as a target for anti-cancer treatment. Multiple studies have shown that inhibition of Kpnβ1 exhibited broad-spectrum cancer killing activities through various mechanisms, including interfering with E2F1 activity (Wang T, Huang Z, Huang N, Peng Y, Gao M, Wang X, et al. Inhibition of KPNB1 inhibits proliferation and promotes apoptosis of chronic myeloid leukemia cells through regulation of E2F1. OncoTargets and therapy. 2019; 12:10455-67), disruption of proteostasis (Zhu Z C, Liu J W, Li K, Zheng J, Xiong Z Q. KPNB1 inhibition disrupts proteostasis and triggers unfolded protein response-mediated apoptosis in glioblastoma cells. Oncogene. 2018; 37(22):2936-52), altering MET proto-oncogene expression and downregulating the epithelial-mesenchymal transition (Zhang Y, Li K F. Karyopherin β1 deletion suppresses tumor growth and metastasis in colorectal cancer (CRC) by reducing MET expression. Biomed Pharmacother. 2019; 120:109127). Most importantly, the impact of Kpnβ1 inhibition showed no toxicity on non-cancer cells, making Kpnβ1 an attractive target for cancer treatment (Van der Watt P J, Maske C P, Hendricks D T, Parker M I, Denny L, Govender D, et al. The Karyopherin proteins, Crm1 and Karyopherin beta1, are overexpressed in cervical cancer and are critical for cancer cell survival and proliferation. Int J Cancer. 2009; 124(8):1829-40). Using an in vitro cervical cancer model, it has been previously demonstrated that siRNA mediated Kpnβ1 inhibition induces various mitotic defects, leading to a G2/M cell cycle arrest and ultimately apoptosis (Angus L, van der Watt P J, Leaner V D. Inhibition of the nuclear transporter, Kpnbeta1, results in prolonged mitotic arrest and activation of the intrinsic apoptotic pathway in cervical cancer cells. Carcinogenesis. 2014; 35(5):1121-31). This further led to the in silico screening, and identification of small molecules, which exhibited nuclear-import inhibitory effect on Kpnβ1 cargoes and reduced cervical and oesophageal tumour growth in xenograft mouse models (US2017/0216285A1, CN10671536). In addition, exogenous Kpnβ1 overexpression rescued the cytotoxic and nuclear import inhibitory effects of the small molecules on cancer cells, confirming that the molecules exert their impact via interfering with Kpnβ1 function (van der Watt P J, Chi A, Stelma T, Stowell C, Strydom E, Carden S, et al. Targeting the nuclear import receptor Kpnbeta1 as an anticancer therapeutic. Mol Cancer Ther. 2016; 15(4):560-73).

Various cancers, and in particular gynaecological cancers, are becoming increasingly resistant or otherwise insensitive to traditional platinum-based chemotherapeutic drugs. As a result, higher dosages may be required for successful anti-cancer therapy. However, most of these chemotherapeutic drugs cause undesirable systemic effects such as cardiac or renal toxicity, alopecia, marrow aplasia, nausea and vomiting. There is therefore a need for new types of therapy that may alleviate at least some of the problems associated with existing approaches to cancer treatment.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with a first aspect of the invention there is provided a combination for use in treating cancer comprising
(a) a therapeutically effective amount of a compound of Formula I:

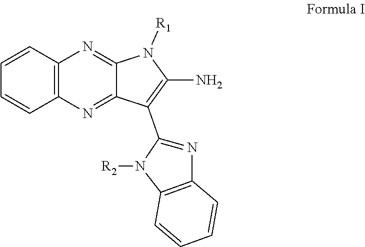

or a pharmaceutically acceptable salt thereof, wherein
   R₁ is a branched or linear C2-C5 alkyl group optionally functionalised with a substituent selected from the group consisting of an amine, an imidazole, an alcohol or a morpholine; and
   R₂ is a hydrogen or a methyl group; and
(b) a therapeutically effective amount of at least one platinum-based chemotherapeutic agent.

R₁ may be selected from:

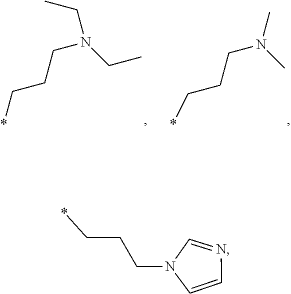

an ethyl group, a propyl group, a butyl group, an iso-butyl group, an iso-pentyl group, a propanol group and

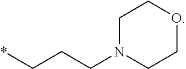 [SMILES: *CCCN1CCOCC1]

The compound of Formula I may be selected from:

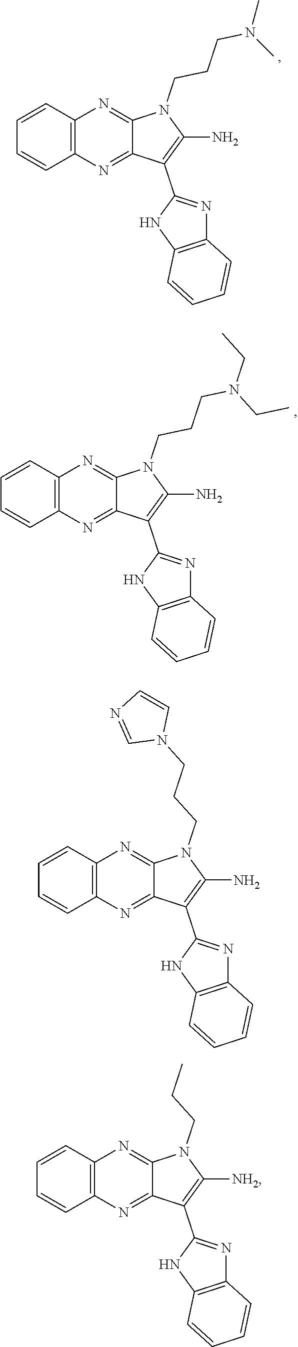

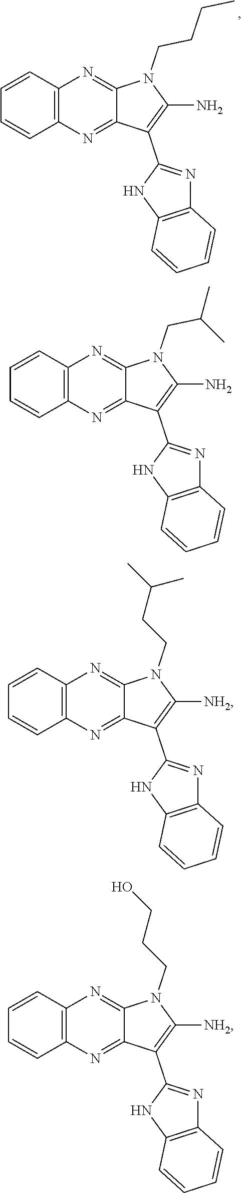

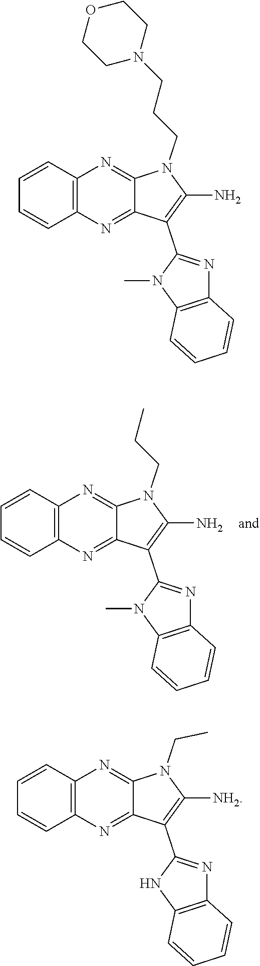 [SMILES: Cn1c(-c2c(N)n(CCCN3CCOCC3)c3nc4ccccc4nc23)nc2ccccc21 ; CCCn1c(N)c(-c2nc3ccccc3n2C)c2nc3ccccc3nc21 ; CCn1c(N)c(-c2nc3ccccc3[nH]2)c2nc3ccccc3nc21]

The compound of Formula I may be

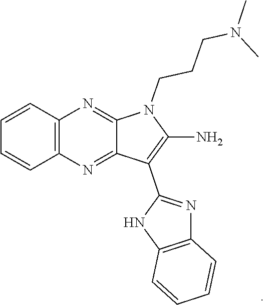 [SMILES: CN(C)CCCn1c(N)c(-c2nc3ccccc3[nH]2)c2nc3ccccc3nc21]

The combination may provide an enhanced therapeutic anti-cancer effect. The combination may provide a synergistic therapeutic anti-cancer effect.

The cancer may be selected from the group consisting of cervical cancer, oesophageal cancer, ovarian cancer, breast cancer and uterine cancer. The at least one platinum-based chemotherapeutic agent may be known to be effective in treating said cancer. The platinum-based chemotherapeutic agent may be selected from the group consisting of cisplatin, carboplatin, oxaliplatin, nedaplatin, lobaplatin and heptaplatin. The platinum-based chemotherapeutic agent may be cisplatin.

In accordance with a second aspect of the invention, there is provided a use of a compound of Formula I:

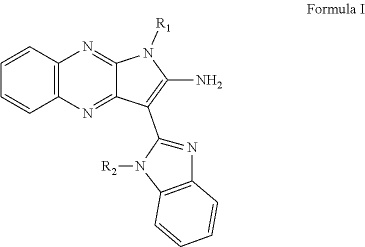

or a pharmaceutically acceptable salt thereof, wherein
   R₁ is a C2-C5 alkyl group, branched or linear, optionally functionalised with a substituent selected from the group consisting of an amine, an imidazole, an alcohol or a morpholine; and
   R₂ is a hydrogen or a methyl group,
in combination with at least one platinum-based chemotherapeutic agent for the treatment of cancer.

In accordance with a third aspect of the invention, there is provided a method for treating cancer comprising administering to a subject in need thereof
(a) a therapeutically effective amount of a compound of Formula I:

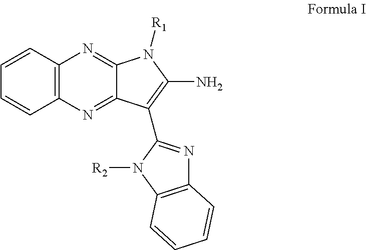

or a pharmaceutically acceptable salt thereof, wherein
   R₁ is a C2-C5 alkyl group, branched or linear, optionally functionalised with a substituent selected from the group consisting of an amine, an imidazole, an alcohol or a morpholine; and
   R₂ is a hydrogen or a methyl group; and
(b) a therapeutically effective amount of at least one platinum-based chemotherapeutic agent to provide a combination therapy having an enhanced therapeutic effect compared to the effect of the compound of Formula I and the at least one platinum-based chemotherapeutic agent administered alone.

In accordance with a fourth aspect of the invention, there is provided a use of a therapeutically effective amount of a compound of Formula I:

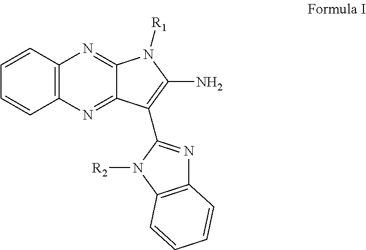

or a pharmaceutically acceptable salt thereof, wherein
   R₁ is a C2-C5 alkyl group, branched or linear, optionally functionalised with a substituent selected from the group consisting of an amine, an imidazole, an alcohol or a morpholine; and
   R₂ is a hydrogen or a methyl group,
for the preparation of a medicament for treating cancer to be administered in combination with at least one platinum-based chemotherapeutic agent, thereby enhancing the anti-cancerous therapeutic effect compared to the effect of each of the medicament comprising the compound of Formula I and the at least one anti-cancer agent administered alone.

The medicament comprising the compound of Formula I may be administered simultaneously with the at least one platinum-based chemotherapeutic agent. Alternatively, the medicament comprising the compound of Formula I and the at least one platinum-based chemotherapeutic agent may be administered sequentially.

In accordance with a fifth aspect of the invention, there is provided a kit for treating cancer comprising
(a) a therapeutically effective amount of a compound of Formula I:

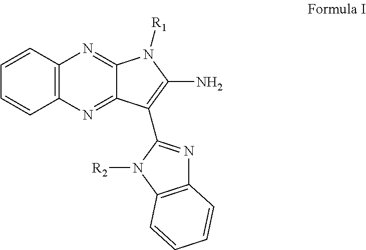

or a pharmaceutically acceptable salt thereof, wherein
   R₁ is a branched or linear C2-C5 alkyl group optionally functionalised with a substituent selected from the group consisting of an amine, an imidazole, an alcohol or a morpholine; and
   R₂ is a hydrogen or a methyl group; and
(b) a therapeutically effective amount of at least one platinum-based chemotherapeutic agent.

Embodiments of the invention will now be described, by way of example only, with reference to the accompanying drawings.

### BRIEF DESCRIPTION OF THE DRAWINGS

In the drawings:

FIG. 1 is a bar graph showing cisplatin IC₅₀ values in cervical cancer cell lines HeLa, CaSki, SiHa and C33A pre-treated with 2.5 μM and 5 μM INI-43 for 2 h, compared to control cells receiving no pre-treatment (Results shown are the mean IC₅₀ value±SEM of three independent experiments (n=6));

FIG. 2 is a bar graph of the relative cell viability following an MTT cell proliferation assay 48 h post-treatment, showing increased cisplatin sensitivity in HeLa, CaSki and SiHa cells after pre-treatment with 5 μM Compound A;

FIG. 3 is a Western blot analysis showing enhanced PARP cleavage in INI-43 and cisplatin combination treated HeLa and SiHa cells (GAPDH was used as a loading control, and quantification via densitometry is shown);

FIG. 4 is a bar graph showing Caspase-3/7 activity in HeLa and SiHa cells was significantly enhanced upon INI-43 and cisplatin combination treatment, compared to cisplatin single treatment (results shown are the mean±SEM of experiments performed in triplicate and repeated three independent times (*p<0.05));

FIG. 5 are graphs of the relative cell viability after cells were subjected to INI-43, cisplatin or the combination treatment for 48 h with viable cells determined using the MTT assay and expressed relative to untreated (arrows indicate enhanced cell death);

FIG. 6 is a plot of the combination index (CI) values against the fraction affected (data shown are the mean±SEM (n=5) and experiments were repeated at least two independent times);

FIG. 7 is a bar graph of the percentage of cell death after cells of cervical cancer cell lines HeLa and Me180 were subjected to INI-43, cisplatin or the combination treatment at a representative concentration;

FIG. 8 is a plot of the combination index (CI) values against the fraction affected for HeLa and Me180 cervical cancer cell lines, where a CI value of more than 1 is antagonistic, equal to 1 is additive, and less than 1 is synergistic;

FIG. 9 is a bar graph of the percentage of cell death after cells of cervical cancer cell lines HeLa and Me180 were subjected to INI-43, cisplatin or the combination treatment at a representative concentration;

FIG. 10 is a plot of the combination index (CI) values against the fraction affected for WHCO6 and KYSE30 oesophageal cancer cell lines, where a CI value of more than 1 is antagonistic, equal to 1 is additive, and less than 1 is synergistic;

FIG. 11 is a Western blot analysis of HeLa and SiHa cells transfected with siRNA for 48 h to confirm Kpnβ1 knock-down, with GAPDH as the loading control;

FIG. 12 is a graph of cisplatin IC₅₀ values determined in Kpnβ1 knock-down HeLa and SiHa cells showing a decrease in cisplatin IC₅₀ in both cell lines when transfected with si-Kpnβ1 compared to si-ctrl (data shown are results±SEM (n=6) of a representative experiment performed two times);

FIG. 13 are bar graphs of relative cell viability from an MTT assay showing that Kpnβ1 knock-down affected cell viability in response to cisplatin treatment in HeLa and SiHa cells (data shown are mean±SEM (n=6) of one representative experiment repeated two times (*p<0.05));

FIG. 14 is a Western blot showing that Kpnβ1 knock-down increased PARP cleavage after cisplatin treatment in HeLa and SiHa cells (GAPDH was included as a loading control, and densitometrical quantification of C-PARP/PARP relative to GAPDH is shown);

FIG. 15 is a Western blot confirming p53 knock-down after SiHa cells were transfected with siRNA for 48 h (GAPDH was included as a loading control);

FIG. 16 is graphs of relative cell viability determined using an MTT assay showing that p53 knock-down does not affect cell viability after 48-h cisplatin treatment (data shown are the mean±SEM of experiments performed in triplicate and repeated two independent times);

FIG. 17 is a Western blot showing that p53 knock-down does not affect cisplatin-induced PARP cleavage in SiHa cells (GAPDH was included as loading control, and densitometrical quantification is presented);

FIG. 18 is a graph of relative cell viability determined with an MTT assay and comparing cell viability between single cisplatin treatment and INI-43-cisplatin combination treatment, in p53 knock-down SiHa cells to compare the degree of enhancement of cell death as a result of the combination treatment (cell viability was normalized to cells receiving single cisplatin treatment, results shown are mean±SEM of experiments performed in triplicate and repeated two independent times (*p<0.05));

FIG. 19 is a Western blot showing that p53 knock-down abrogated the enhancement of PARP cleavage observed after combination treatment compared to single cisplatin treatment (GAPDH was included as the loading control, and densitometrical quantification is shown);

FIG. 20 is a Western blot of p53 content after SiHa cells were treated with DMSO or 5 μM INI-43 for 2 h (a) followed by 50 μg/mL CHX treatment; protein was harvested at the indicated time points (GAPDH served as the loading control);

FIG. 21 is a Western blot of p53 content after SiHa cells were transfected with si-ctrl or si-Kpnβ1 for 48 h; protein was harvested at the indicated time points (GAPDH served as the loading control);

FIG. 22 is a bar graph showing the fold increase in p53 half-life as the mean±SEM from the three independent experiments (*p<0.05);

FIG. 23 is a bar graph showing p53 reporter activity is increased upon 24 h 5 μM INI-43 treatment of SiHa cells (*p<0.05);

FIG. 24 is a bar graph showing p53 reporter activity is enhanced in INI-43-cisplatin combination treated SiHa cells, compared to cells treated with cisplatin alone (*p<0.05);

FIG. 25 is a Western blot showing levels of p53 after single and combination treatment (β-tubulin served as a loading control);

FIG. 26 is a Western blot showing levels of p53 targets p21 and Mcl-1 after single and combination treatment (β-tubulin served as a loading control for Mcl-1 and GAPDH for p2);

FIG. 27 are photographs of the distribution of NFκB subunits (a) p50 or (b) p65 analysed by immunofluorescence after single (30 μM cisplatin) or combination treatment

FIG. 28 are bar graphs of the fluorescence intensities quantified using ImageJ and expressed as nuclear fluorescence relative to cytoplasmic fluorescence (Fc (Nu/Cy)) for p50 (a) and p65 (b) (results shown are mean±SEM of 6 cells (*p<0.05, a, b);

FIG. 29 is a Western blot analysis showing increased nuclear p50 and p65 levels after cisplatin treatment, which was reduced if cells were pre-treated with INI-43 (TBP served as the nuclear loading control and β-tubulin to confirm that pure nuclear lysates were obtained in comparison to a random cytoplasmic protein sample ‘C’);

FIG. 30 is a bar graph of a p65 reporter assay showing increased p65 luciferase activity after 30 μM single cisplatin treatment, which was reduced with INI-43 pre-treatment (*p<0.05); and

FIG. 31 is a Western blot showing changing levels of various NFκB targets (cyclin D1, c-Myc and XIAP) after single or combination treatment, and enhanced phosphorylation of H2A.X (γH2AX) in combination treated cells, indicative of increased DNA damage (β-tubulin was included as the loading control).

### DETAILED DESCRIPTION WITH REFERENCE TO THE DRAWINGS

Combined use of small molecule nuclear import inhibitors that bind and inhibit the Kpnβ1 transport protein with platinum-based chemotherapeutic agents result in enhanced and/or synergistic anti-cancer effects in cancer cells. Without wishing to be bound by any particular theory or mechanism of action this enhanced and/or synergistic effect may be attributed to enhanced cell death mediated through p53 and NFκB function.

The small molecule inhibitors described herein were initially identified from an in silico screening for binding affinity to Kpnβ1. Subsequent studies have resulted in substituted pyrrolo[2,3-b]quinoxalines of the Formula I:

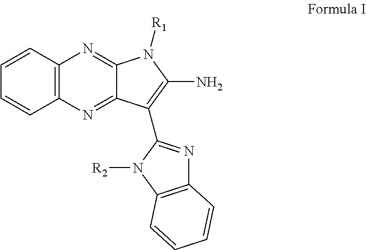

or a pharmaceutically acceptable salt thereof, wherein R₁ is a branched or linear C2-C5 alkyl group optionally functionalised with a substituent selected from the group consisting of an amine, an imidazole, an alcohol or a morpholine; and R₂ is a hydrogen or a methyl group, being identified as inhibitors of the Kpnβ1 transport protein displaying anti-cancer cell killing effects. It has now been surprisingly found that these inhibitors of Kpnβ1 can also be used in combination with clinically relevant platinum-based chemotherapeutic agents such as cisplatin, carboplatin, oxaliplatin, nedaplatin, lobaplatin and heptaplatin to treat cancer. Such combined use may produce greater anti-cancer effects compared to the effect of each when used individually, and in particular may produce greater anti-cancer effects compared to the additive effects of each used individually.

The terms “treating”, “treatment” and the like are used herein to mean affecting a subject, tissue, or cell to obtain a desired pharmacological and/or physiological effect. The effect may be therapeutic in terms of a partial or complete cure of the cancer. “Treating” as used herein covers any treatment of cancer in a subject; inhibiting the cancer, i.e., arresting its development; or relieving or ameliorating the effects of the cancer, i.e., cause regression of the tumour or of the effects of the cancer.

The term “subject” as used herein refers to any mammal having cancer which requires treatment, typically a human.

The term “anti-cancer” as used herein in reference to “anti-cancer agent”, “anti-cancer therapeutic effect” “anti-cancerous effect” and the like is meant in its broadest scope as in known in the art, and includes the activities of arrest of cell growth, induction of apoptosis, induction of differentiation, cell death and the like.

As used herein the terms “therapeutically effective amount” or “effective amount” refers to an amount of the compound of Formula I as defined above and/or the platinum-based chemotherapeutic agent that is effective in treating cancer. The specific “effective amount” will vary according to the particular condition being treated, the physical condition and clinical history of the subject, the duration of the treatment and the nature of the combination of agents applied and its specific formulation.

The terms “enhanced effect” or “greater effect” is used herein to refer to a combined effect caused by the combined action and/or interaction between the compound of Formula I and at least one other platinum-based chemotherapeutic agent wherein the observed effect (e.g., cytotoxicity) in the presence of the drugs together is significantly higher than the effect of each individual drug (e.g., cytotoxicities) administered separately. In some embodiments, the observed combined effect of the drugs is significantly higher than each of the individual effects. In certain embodiments the term significant means that the observed p<0.05.

The terms “synergistic” and “synergism” are used herein to refer to a combined effect caused by the combined action and/or interaction between the compound of Formula I and at least one other platinum-based chemotherapeutic agent wherein the observed effect (e.g., cytotoxicity) in the presence of the drugs together is higher than the sum of the individual effects (e.g., cytotoxicities) of each drug administered separately. In some embodiments, the observed combined effect of the drugs is significantly higher than the sum of the individual effects. In certain embodiments the term significant means that the observed p<0.05.

As used herein, the term “platinum-based chemotherapeutic agent” refers to platinum-containing compounds or complexes which act to impair cell function by forming covalent bonds with amino, carboxyl, sulfhydryl and/or phosphate groups in biologically important molecules. The most important sites of binding are DNA, RNA and proteins. Platinum-containing compounds are believed to induce apoptotic cell death by inhibiting transcription. When RNA polymerases transcribe DNA they stall at the platinum-adduct formed and recruit transcription-coupled repair machinery. If this machinery fails to repair the lesion the cell evokes a programmed cell death pathway.

Accordingly, a combination for use in treating cancer is provided comprising (a) a therapeutically effective amount of the quinoxaline derivatives of the Formula I:

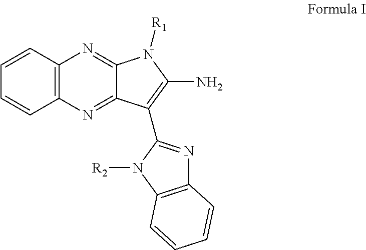

or a pharmaceutically acceptable salt thereof, wherein R₁ is a branched or linear C2-C5 alkyl group optionally functionalised with a substituent selected from the group consisting of an amine, an imidazole, an alcohol or a morpholine; and R₂ is a hydrogen or a methyl group; and (b) a therapeutically effective amount of at least one additional platinum-based chemotherapeutic agent.

R₁ in Formula I may be selected from:

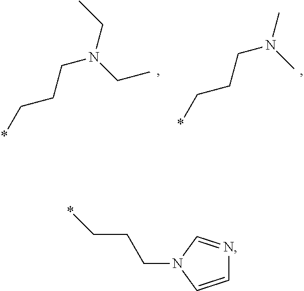

an ethyl group, a propyl group, a butyl group, an iso-butyl group, an iso-pentyl group, a propanol group and

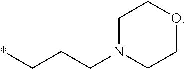 [SMILES: *CCCN1CCOCC1]

More particularly, the compound of Formula I may be selected from the following group of quinoxaline derivatives:

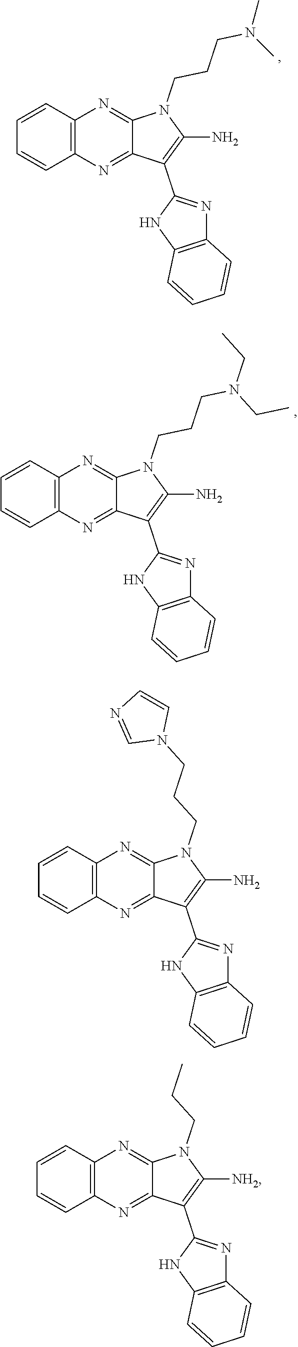

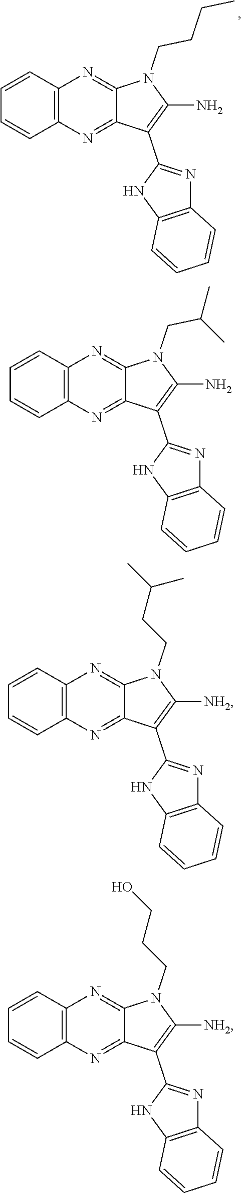

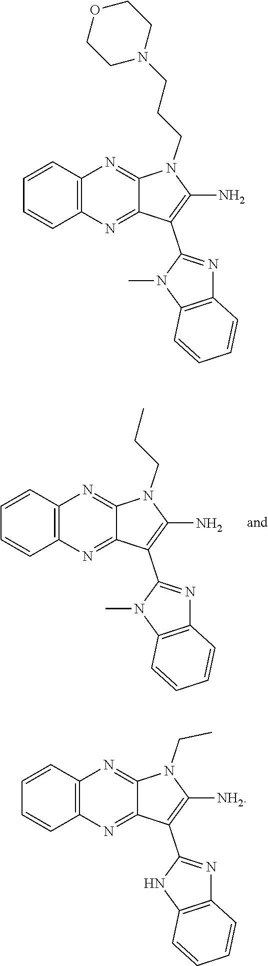 [SMILES: Cn1c(-c2c(N)n(CCCN3CCOCC3)c3nc4ccccc4nc23)nc2ccccc21 ; CCCn1c(N)c(-c2nc3ccccc3n2C)c2nc3ccccc3nc21 ; CCn1c(N)c(-c2nc3ccccc3[nH]2)c2nc3ccccc3nc21]

Preferably, the compound of Formula I is

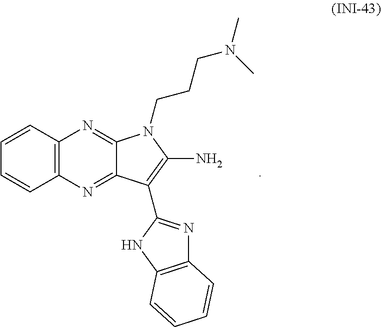 [SMILES: CN(C)CCCn1c(N)c(-c2nc3ccccc3[nH]2)c2nc3ccccc3nc21]

Use of the compound of Formula I as defined above in combination with at least one additional platinum-based chemotherapeutic agent for the treatment of cancer and methods for treating cancer with a combination of (a) a therapeutically effective amount a compound of Formula I and (b) at least one additional platinum-based chemotherapeutic agent are further are also provided.

With such use and in a method of treating cancer, the combination therapy (CT) has an enhanced therapeutic effect compared to the effect of the compound of Formula I and the at least one additional platinum-based chemotherapeutic agent administered alone.

Determining the dosage and duration of treatment is within the skillset of those proficient in the art. For example, dosages of the compounds or agents are suitably determined depending on the individual cases taking symptoms, age and sex of the subject and the like into consideration. The amount of the compound to be incorporated into a pharmaceutical composition varies with dosage route, solubility of the compound, administration route, administration scheme and the like. An effective amount for a particular patient may vary depending on factors such as the condition being treated, the overall health of the patient and the method, route and dose of administration. The clinician using parameters known in the art makes determination of the appropriate dose. Generally, the dose begins with an amount somewhat less than the optimum dose and it is increased by small increments thereafter until the desired or optimum effect is achieved.

The compound of Formula I as defined above may be administered in an amount sufficient to allow the reduction of the normal dose of the at least one platinum-based chemotherapeutic agent required to effect the same degree of treatment by at least any of 5%, 10%, 20%, 30%, 50%, 60%, 70%, 80%, 90%, or more.

The compound of Formula I as defined above may be administered in an amount of 0.1 mg/kg body weight to 100 mg/kg body weight, preferably 0.1 mg/kg to 50 mg/kg as may be found to be appropriate. Lower or higher concentrations may be used, depending on the type, number and amount of platinum-based anti-cancer agent co-administered to a subject, the subject to be treated (age, gender, weight etc.), the type of cancer to be treated and the stage of the disease. As exemplified hereinbelow, the combination of a compound of Formula I and at least one platinum-based chemotherapeutic agent significantly reduces the survival of cancer cell lines. The inhibiting activity of the exemplary combination was significantly higher as compared to the additive inhibition activity of each of the components of the combination, and thus defined as synergistic effect.

Each of component (a) and (b) of the combination as defined above may be administered simultaneously (concurrently) or sequentially (separately) to treat cancer. When the components are administered simultaneously, the compound of Formula I and the at least one platinum-based chemotherapeutic agent may be contained in the same composition (e.g., a composition comprising both the compound of Formula I and the at least one platinum-based chemotherapeutic agent together with a pharmaceutically acceptable excipient or diluent or the like) or in separate compositions administered shortly (within minutes) after one another. With sequential administration the compound of Formula I and the at least one platinum-based chemotherapeutic agent are administered with a time separation. The time separation may be 15 minutes or more and either the compound of Formula I or the platinum-based chemotherapeutic agent may be administered first, but preferably the compound of Formula I is administered first. The components of the combination will then be in separate compositions.

Each of the above-described combinations may provide an enhanced therapeutic anti-cancer effect. In particular, the combinations may provide a synergistic therapeutic anti-cancer effect. The combinations may find use in treating a wide range of different types of cancers and typically those that are presently being treated with platinum-based chemotherapeutic drugs or other combination therapies employing platinum-based chemotherapeutics. In particular, the cancer to be treated with the combination may be selected from the group consisting of cervical cancer, oesophageal cancer, ovarian cancer, breast cancer and uterine cancer.

The at least one additional platinum-based chemotherapeutic agent to be used in combination with the small molecule inhibitors may be known to be effective in treating a selected type of cancer. Traditional platins The platinum-based chemotherapeutic agent may be selected from the group consisting of cisplatin, carboplatin, oxaliplatin, nedaplatin, lobaplatin and heptaplatin. The platinum-based chemotherapeutic agent may be cisplatin.

A therapeutically effective amount of a compound of Formula I as defined above may also be used for the preparation of a medicament for treating cancer to be administered in combination with the at least one additional platinum-based chemotherapeutic agent, thereby enhancing the anti-cancerous therapeutic effect compared to the effect of each of the medicament comprising the compound of Formula I and the at least one additional anti-cancer agent administered alone. As previously mentioned with respect to the method of treatment, the medicament comprising the compound of Formula I may be administered simultaneously with the at least one additional platinum-based chemotherapeutic agent. The medicament may be in the form of two separate compositions, each containing one of the components of the combination or a single composition containing both active components of the combination. Alternatively, the medicament comprising the compound of Formula I and the at least one additional platinum-based chemotherapeutic agent may be administered sequentially and be in the form of separate pharmaceutical compositions. Such medicaments can have any suitable form and may include other ingredients including one or more pharmaceutically acceptable excipients, diluents, adjuvants and/or carriers.

A kit for treating cancer comprising (a) a therapeutically effective amount of a compound of Formula I as defined above and (b) a therapeutically effective amount of at least one additional platinum-based chemotherapeutic agent is also provided. Each of the components (a) or (b) may be formulated with at least one pharmaceutically acceptable adjuvant such as a diluent or carrier. Each of the components (a) or (b) may be adapted for administration simultaneously or sequentially.

To demonstrate the enhanced and/or synergistic effect seen with combined use of nuclear import inhibitors and platinum-based chemotherapeutic agents to kill cancer cells of various cancer cell lines, the following experiments were carried out with a combination of the quinoxaline derivative, referred to hereinbelow as INI-43:

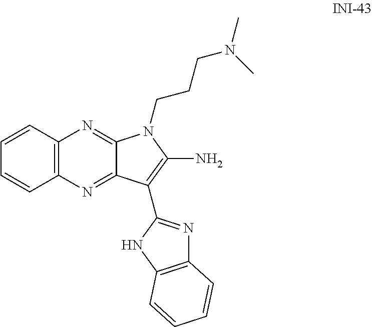 [SMILES: CN(C)CCCn1c(N)c(-c2nc3ccccc3[nH]2)c2nc3ccccc3nc21]

and cisplatin.

### Experimental Section

### Methods

### Cell Lines and Tissue Culture

HeLa, SiHa, CaSki and C₃₃A cell lines were purchased from the American Type Culture Collection (ATCC) and maintained in Dulbecco's Modified Eagle's Medium (DMEM, Gibco, Life Technologies) containing 10% Fetal Bovine Serum (Gibco, Life Technologies), supplemented with 100 U/mL penicillin and 100 μg/mL streptomycin. Cells were cultured at 37° C. in a humidified chamber with 5% CO₂. All cell lines were authenticated by DNA profiling using the Cell ID System (Promega, Madison, WI, USA).

### Half Inhibitory Concentration (IC₅₀) Determination

Cells were plated in 96-well plates and subjected to single or CT (2 h INI-43 pre-treatment followed by cisplatin treatment, without removing IN1-43 from the media) for 48 h. Following treatment, MTT (Sigma) was added and 4 h later crystals solubilized using 10% SLS in 0.01 M HCl. Absorbencies were measured at 595 nm and IC₅₀ values determined via plotting [Fa/(1-Fa)] in log scale against log cisplatin concentration, where:

$$Fa = \frac{100 - \% viable cells relative to the untreated}{100}$$

The half inhibitory concentration was calculated using the formula IC₅₀=10^{x-intercept}

### Drug Washout Experiments

Cells were plated in 96-well plates and subjected to 2 h INI-43 pre-treatment followed by cisplatin treatment, with or without removing INI-43 from the media, for 48 h. Following treatment, MTT (Sigma) was added and 4 h later crystals solubilized using 10% SLS in 0.01 M HCl.

### Caspase-3/7 Assay

Cells were subjected to single or CT for 48 h, and caspase-3/7 activity monitored using the Promega Caspase-Glo^{R} 3/7 assay, according to the manufacturer's instructions. Luminescence was measured using the Veritas™ microplate luminometer (Promega) and results standardized to viable cells in each treatment as determined by MTT assays performed in parallel.

### Combination Index (CI) Determination

To elucidate the nature of the combined use of INI-43 and cisplatin, the Chou-Talalay method was adopted (Chou T C, Talalay P. Quantitative analysis of dose-effect relationships: the combined effects of multiple drugs or enzyme inhibitors. Adv Enzym Regul. 1984; 22:27-55). Cell viability was determined after 48-h treatment at fixed INI-43 to cisplatin ratios of 1:3, 1:4 and 1:5 (Table 1 below). Cell viability was converted to fraction affected (Fa) and CI calculated using CompuSyn software (ComboSyn, Inc.).

TABLE 1
Cisplatin and INI-43 concentrations used in the combination
index determination experiment. Cells were treated
with cisplatin only, INI-43 only or a combination of
the two using the concentrations indicated below.

| | INI-43 (μM) | | |
|---|---|---|---|
| | 1:3 | 1:4 | 1:5 |
| Cisplatin (μM) | INI-43:cisplatin | INI-43:cisplatin | INI-43:cisplatin |
| 3 | 1.00 | 0.75 | 0.60 |
| 7.5 | 2.50 | 1.88 | 1.50 |
| 15 | 5.00 | 3.75 | 3.00 |
| 22.5 | 7.50 | 5.63 | 4.50 |
| 30 | 10.00 | 7.50 | 6.00 |
| 45 | 15.00 | 11.25 | 9.00 |

siRNA Transfection

Cells were transfected using Transfectin (Bio-Rad) and 20 nM si-Kpnβ1 (H-7, sc-35,736, Santa Cruz) or 30 nM si-p53 (sc-29,435, Santa Cruz). Control cells were transfected with the equivalent amount of ctrl siRNA (si-ctrl, sc-37,007, Santa Cruz).

### Western Blot Analysis

For protein extraction, cells were washed with PBS and lysed using RIPA buffer (50 mM Tris-CI, pH 7.4, 150 mM NaCl, 1% (w/v) sodium deoxycholate, 0.1% (v/v) SDS, 1% (v/v) Triton X-100, 2 mM EGTA, 2 mM EDTA, 50 mM NaF, 5 mM Na₂P2O₇, 1× complete protease inhibitor cocktail (Roche) and 0.1 M Sodium Orthovanadate). For PARP cleavage analysis, dead cells were collected by centrifugation and combined with live cell lysates. Lysates were sonicated, centrifuged, and the supernatant quantified using the BCA Protein Assay Kit (Pierce, Thermo Scientific) according to the manufacturer's instructions. Proteins were subjected to Western blot analysis using the following antibodies: rabbit anti-Kpnβ1 (H-300, sc-11,367, Santa Cruz), rabbit anti-β-tubulin (H-235, sc-9104, Santa Cruz), rabbit anti-PARP1/2 antibody (H-250, sc-7150, Santa Cruz), mouse anti-GAPDH (0411, sc-47,724, Santa Cruz), rabbit anti-p21 (H-164, sc-756, Santa Cruz), rabbit anti-Mcl-1 (H-260, sc-20,679, Santa Cruz), mouse anti-cyclin D1 (HD11, sc-246, Santa Cruz), rabbit anti-c-Myc (N-262, sc-764, Santa Cruz), mouse anti-p53 (DO-7, M7001, DakoCytomation), mouse anti-XIAP (610,763, BD Biosciences), and rabbit anti-phospho-Histone H2AX (γH2AX, Ser139, 20E3, #9718, Cell Signal).

### Nuclear/Cytoplasmic Fractionation

For nuclear/cytoplasmic protein extraction, cells were collected by trypsinization. Cell pellets were resuspended in 10 mM HEPES (pH 7.9), 50 mM NaCl, 0.5 M sucrose, 0.1 mM EDTA and 0.5% Triton X-100, followed by centrifugation at 1000×G for 10 min to separate cytoplasmic (supernatant) and nuclear fractions (pellet). Cytoplasmic fractions were centrifuged at 14 000× G for 15 min at 4° C., and the supernatant stored at −80° C. Nuclear pellets were washed in 10 mM HEPES, 10 mM KCl, 0.1 mM EDTA and 0.1 mM EGTA, and centrifuged at 1000×G for 5 min. Pellets were then resuspended in 10 mM HEPES (pH 7.9), 500 mM NaCl, 0.1 mM EDTA, 0.1 mM EGTA and 1% (v/v) NP-40 and vortexed for 15 min at 4° C. to extract the nuclear protein, followed by centrifugation at 14 000× G for 10 min. The fractions were quantified using the BCA Protein Assay Kit (Pierce, Thermo Scientific) according to the manufacturer's instructions, followed by western blot analysis.

### p53 Half-Life (T_{1/2}) Determination

Cells were treated with 5 μM INI-43 or DMSO for 2 hours or transfected with 20 nM si-ctrl or si-Kpnβ1 for 48 h prior to p53 half-life determination. Cells were treated with 50 μg/mL cycloheximide (CHX), and protein harvested at 0, 15, 30, 45, 60 and 90 min after CHX treatment. p53 content was analysed by western blotting. Bands were quantified by densitometrical scanning using ImageJ, normalised to GAPDH and expressed as a value relative to p53 intensity at time 0. Relative band intensities were plotted in log scale against time of CHX treatment and a linear trendline drawn. The half-life was calculated using the formula T_{1/2} (minutes)=Log(2)/[slope].

### Immunofluorescence

SiHa cells were plated on glass coverslips and treated for 24 h before fixation with 4% paraformaldehyde. Cells were permeabilised using 0.5% Triton-X-100/PBS and blocked using 1% BSA/PBST with 0.3 M Glycine. Primary antibody incubations were performed in 1% BSA/PBST, followed by secondary antibody incubation (Cy3 conjugated goat anti-rabbit, Jackson ImmunoResearch). Cell nuclei were counterstained with 0.5 μg/mL DAPI, and images captured using the Zeiss inverted fluorescence microscope under 100× oil immersion.

### Luciferase Reporter Assay

SiHa cells were transfected with 100 ng p65-luciferase reporter construct (containing five copies of the p65-binding site, Promega) or 200 ng p53-luciferase reporter construct (containing thirteen wildtype p53 binding sites, Addgene plasmid #16442, Addgene Plasmid Repository (el-Deiry W S, Tokino T, Velculescu V E, Levy D B, Parsons R, Trent J M, et al. WAF1, a potential mediator of p53 tumor suppression. Cell. 1993; 75(4):817-25) and 10 ng pRL-TK (encoding Renilla luciferase, Promega), using Genecellin transfection reagent (Celtic Molecular Diagnostics). The following day cells were treated with 5 μM INI-43 for 2 h, followed by 30 μM cisplatin for 24 h, and luciferase activity assayed using the Dual-Luciferase Report assay system (Promega), according to the manufacturer's instructions. Luciferase readings were measured using the Veritas™ microplate luminometer (Promega) and normalised to Renilla luciferase from the same extract.

### Statistical Analysis

For all data comparisons, the Student's t test was performed using Microsoft Excel. A p value of <0.05 was considered statistically significant.

### Results

### INI-43 Pre-Treatment Enhanced HeLa and SiHa Cell Sensitivity to Cisplatin

To determine if nuclear import inhibition influences cancer cell sensitivity to cisplatin treatment, cisplatin IC₅₀ values were compared between cervical cancer cells with and without INI-43 pre-treatment. Pre-treatment was conducted at sublethal INI-43 concentrations (≤10 μM) for 2 h (concentrations which were previously shown to reduce nuclear import of various Kpnβ1 cargoes (van der Watt et al. Mol Cancer Ther. 2016; 15(4):560-7)), followed by cisplatin treatment.

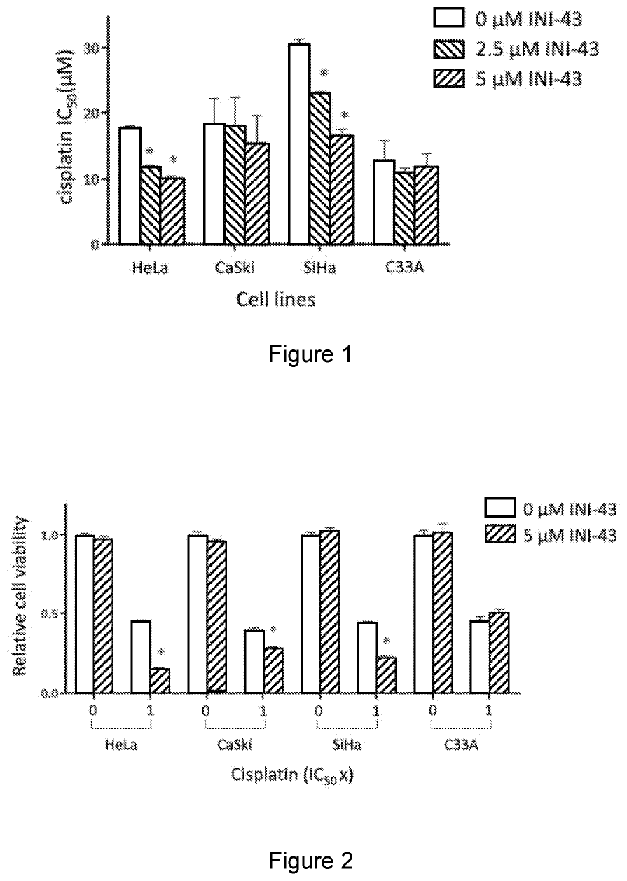

Cisplatin IC₅₀ values after 48-h treatments were 18.0 μM, 18.1 μM, 30.8 μM and 12.8 μM for HeLa, CaSki, SiHa and C33A, respectively. However, when cells were pre-treated with INI-43, a significant dose-dependent decrease in cisplatin IC₅₀ was observed in both HeLa and SiHa cells (44 and 46% in HeLa and SiHa cells, respectively) (FIG. 1). A small reduction in cisplatin IC₅₀ was observed in CaSki cells and no change in cisplatin IC₅₀ observed in C33A cells.

Cell viability was next examined at fixed cisplatin concentrations, with or without INI-43 pre-treatment. FIG. 2 shows that in HeLa, CaSki and SiHa cells, CT resulted in significantly decreased cell viability compared to their cisplatin-only treated counterparts. In line with the cisplatin IC₅₀ results, C33A showed no change in cell viability after single or CT. As 5 μM INI-43 on its own did not affect cell viability across all cell lines, this suggests that the enhanced cell death observed in the CT was due to the combined action of INI-43 and cisplatin, rather than addition of independent effects of the two drugs.

Since INI-43 was not removed from the cells before cisplatin treatment it was next determined whether the effects of INI-43 would be sustained following drug removal, or whether the INI-43 treatment effects were transient. Washout experiments were performed where cells were incubated with INI-43 for 2 h, and thereafter either treated with cisplatin (with INI-43 still present), treated with cisplatin after INI-43 removal (washout 1), or treated with cisplatin 2 h after INI-43 removal (washout 2). Results showed that even after INI-43 was removed before cisplatin treatment there was still significantly reduced cell viability in response to the combination treatment when compared to the effects of cisplatin alone, suggesting that the effects of INI-43 are not reversible following drug washout. The enhancement of cell death upon CT was slightly reduced in HeLa cells after INI-43 washout, but this is likely due to the rapid doubling time of HeLa cells, and thus quick synthesis of nascent Kpnβ1 which would begin to counteract the effects of INI-43 over time.

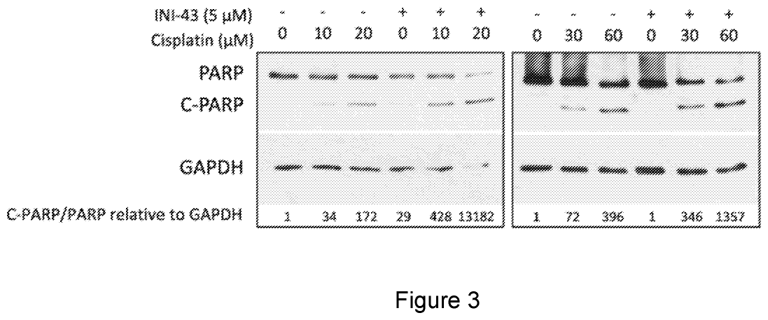

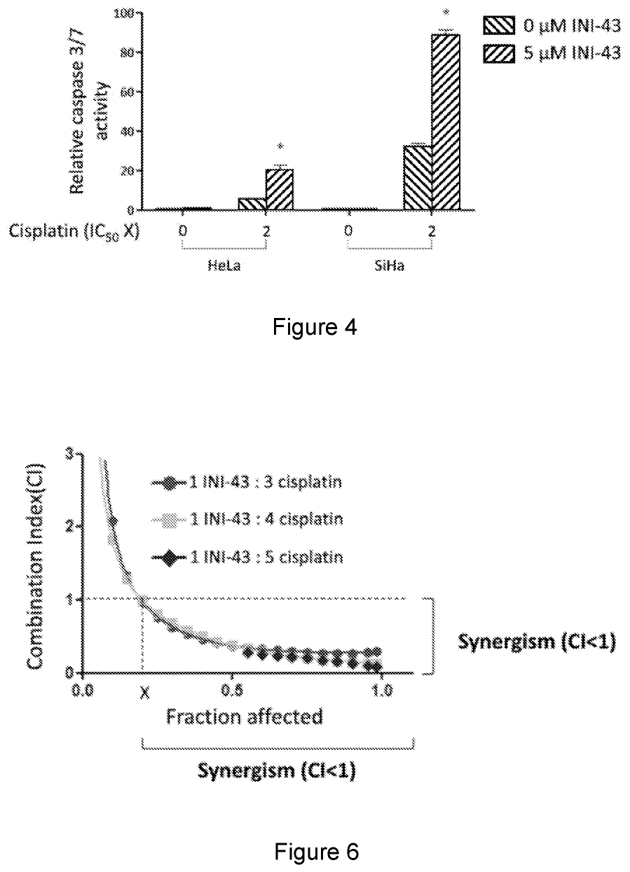

To determine whether INI-43-cisplatin CT resulted in increased apoptosis, PARP cleavage and caspase-3/7 activation were assayed. Protein from live and dead cells was collected and PARP status examined by western blot. In both HeLa and SiHa cells, enhanced PARP cleavage was observed in the combination treated cells compared to those receiving cisplatin only (FIG. 3). Supporting the cell viability data, 5 μM INI-43 treatment on its own showed negligible apoptosis. Investigation of caspase-3/7 activation revealed that combination treated cells exhibited increased caspase-3/7 activation compared to cisplatin only treated cells (3.6-fold and 2.8-fold increase in HeLa cells and SiHa cells, respectively) (FIG. 4). These results suggest that nuclear import inhibition via INI-43 pre-treatment sensitized both HeLa and SiHa cells to cisplatin through enhanced activation of apoptosis.

### INI-43 and Cisplatin Combination Treatment Resulted in Synergistically Enhanced Cell Death in Cervical Cancer Cell Lines

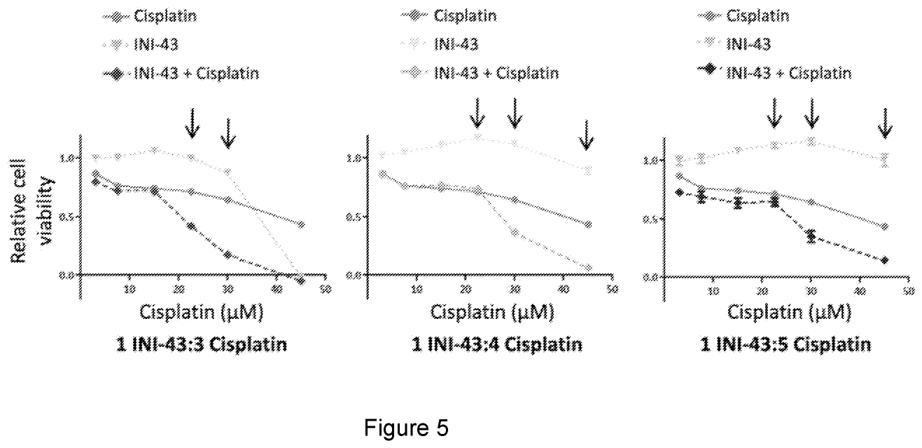

Since the concentration of INI-43 used was not sufficient to induce significant cell death alone, and yet in combination with cisplatin it significantly enhanced cell death, it was proposed that INI-43 and cisplatin engaged in a synergistic interaction, where the cytotoxic effect of their combined use was greater than the additive effects of either drug used independently. To test this, the combination index (CI) was evaluated, according to the Chou-Talalay method, using a fixed dose ratio. SiHa cells were treated with INI-43 and cisplatin at varying concentrations to give INI-43-to-cisplatin ratios of 1:3, 1:4 or 1:5 (Table 1 above). Cells were pre-incubated with respective INI-43 concentrations for 2 h prior to cisplatin treatment. Results showed that while cisplatin reduced cell viability in a dose-dependent manner, pre-treatment with INI-43 significantly enhanced this effect (FIG. 5). Based on the cell viability results, the CI values were calculated using CompuSyn software (ComboSyn, Inc.) and plotted against Fraction Affected (Fa), where Fa=0 and Fa=1 equates to no cell death and complete cell death, respectively. At Fa>0.2, CI values were below 1 for INI-43 to cisplatin ratios of 1:3, 1:4 and 1:5, revealing synergistically enhanced cell death (FIG. 6).

### INI-43 and Cisplatin Combination Treatment Resulted in Synergistically Enhanced Cell Death in Cervical and Oesophageal Cancer Cell Lines

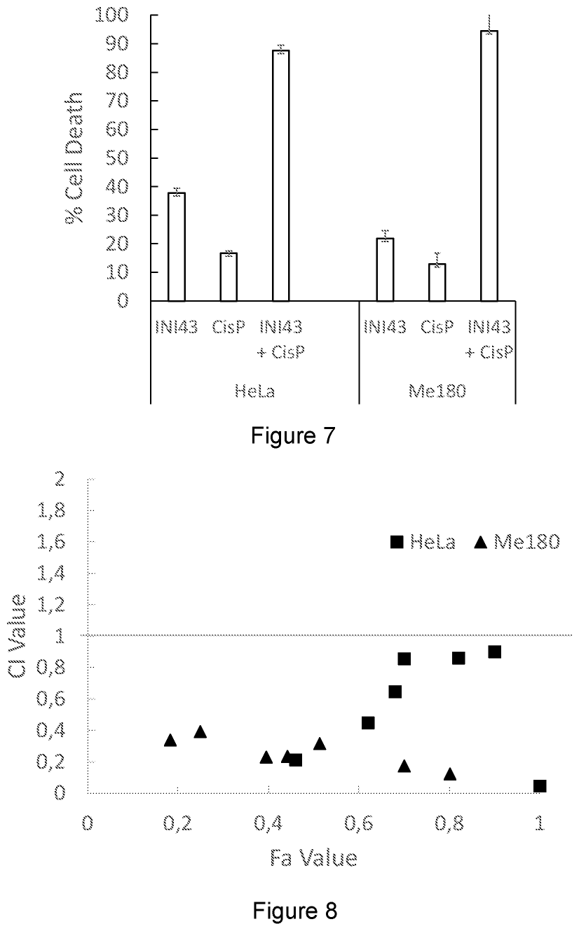

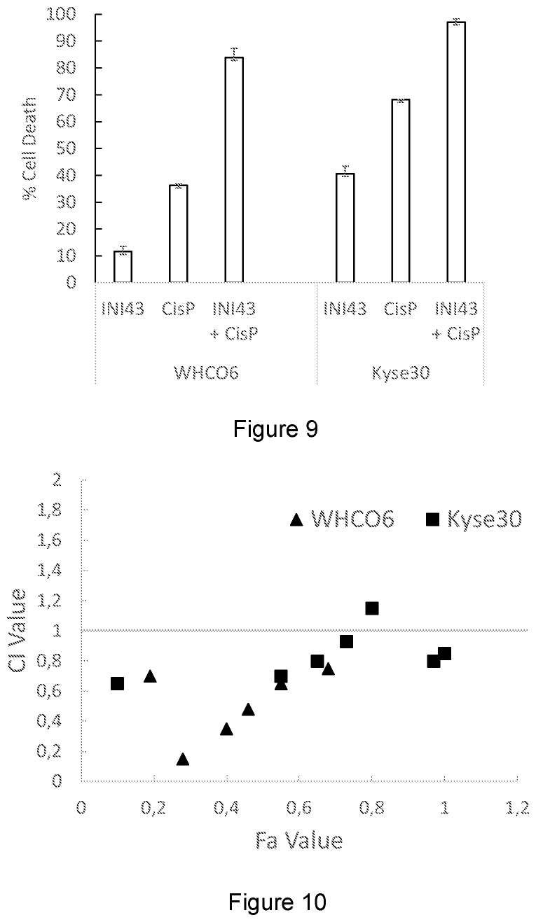

Combination treatment of INI-43 and cisplatin also showed synergism in the cervical cancer cell lines, HeLa and Mel 80, and the oesophageal cancer cell lines WHC05 and Kyse 30. Cell viability results and CI values for the cervical cancer cell lines are shown in FIGS. 7 and 8. Cell viability results and CI values for the oesophageal cancer cell lines are shown in FIGS. 9 and 10.

Kpnβ1 knock-down sensitized cervical cancer cells to cisplatin

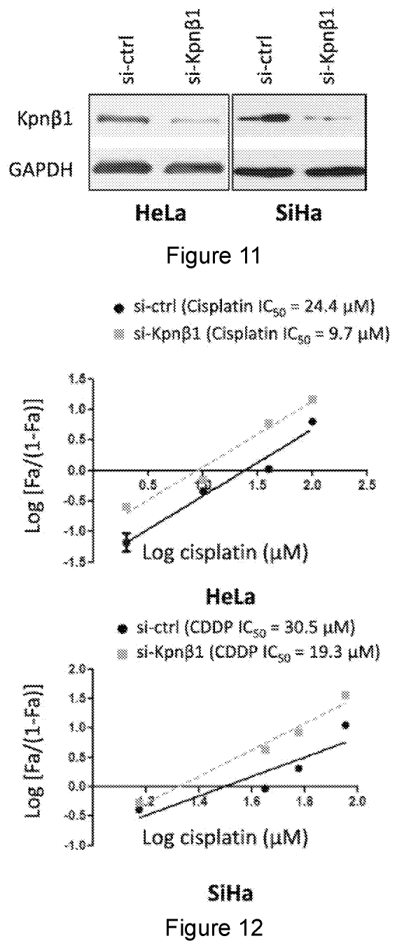

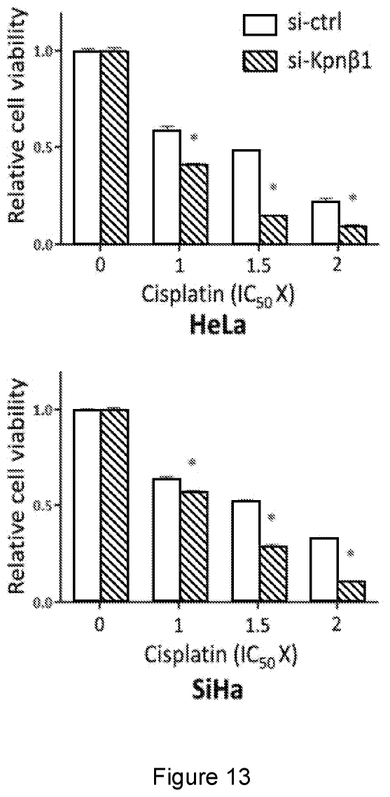

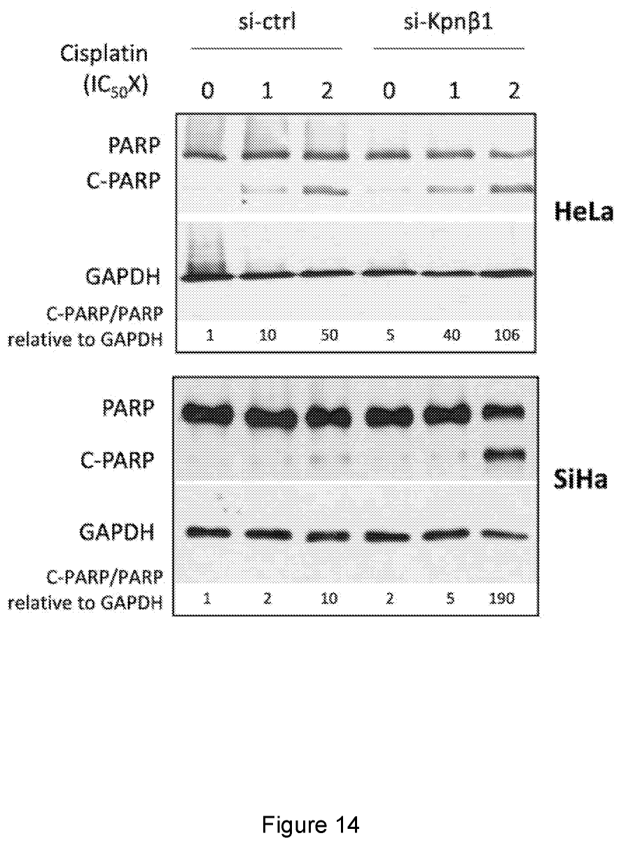

To confirm that the enhancing effect of INI-43 on cisplatin cytotoxicity was due specifically to nuclear import inhibition, rather than off-target effects, cisplatin sensitivity was examined in Kpnβ1 knock-down cells. Cells were transfected with Kpnβ1 targeting siRNA (si-Kpnβ1) or control siRNA (si-ctrl), and cisplatin sensitivity determined. Successful Kpnβ1 knock-down was confirmed by western blotting 48 h post transfection, at which point cisplatin treatment began (FIG. 11). In Kpnβ1 knock-down cells, there was a significant reduction of cisplatin IC50 from 24.4 μM in the control cells to 9.7 μM in HeLa cells, and 30.5 μM in the control cells to 19.3 μM in SiHa cells (FIG. 12). To confirm this effect, cell viability was measured after cisplatin treatment in Kpnβ1 knock-down and control siRNA transfected cells. To eliminate the cell death that was caused by Kpnβ1 knock-down, cell viability was normalized to untreated cells in each transfection group. Results indicated that Kpnβ1 knock-down cells were more sensitive to cisplatin-induced cell death at all concentrations tested (FIG. 13). Furthermore, Kpnβ1 knock-down HeLa and SiHa cells exhibited visibly increased PARP cleavage after cisplatin treatment compared to control siRNA-transfected cisplatin-treated cells (FIG. 14). Collectively, these results show that Kpnβ1 knock-down enhanced sensitivity to cisplatin, similarly to that observed after INI-43 treatment, supporting that INI-43 increases cisplatin sensitivity by disrupting Kpnβ1 function.

### p53 is an Important Mediator of INI-43-Cisplatin-Induced Cell Death

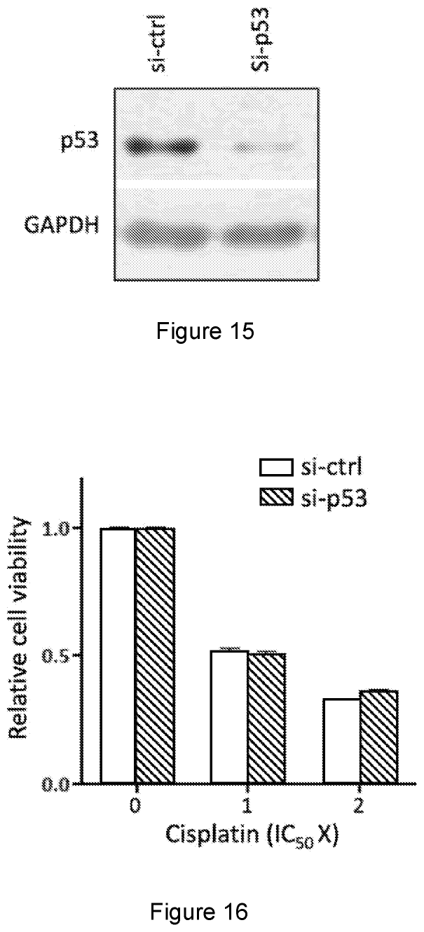

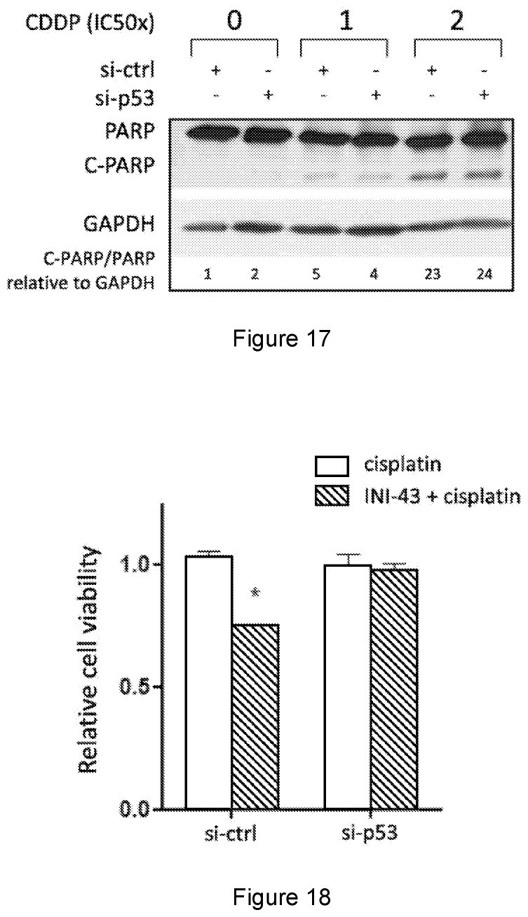

To elucidate whether p53 might play a role in the cellular response to cisplatin and furthermore, whether the enhanced cisplatin sensitivity in INI-43 pre-treated cells involved p53, the effects of p53 knock-down were examined. p53 knock-down was confirmed via western blot 48 h post transfection, at which point cells were subjected to drug treatments as previously described (FIG. 15). After single cisplatin treatment, p53 knock-down cells exhibited similar cell viability to si-ctrl transfected cells, suggesting that p53 was not involved in cisplatin-induced cell death (FIG. 16). These results were validated by PARP cleavage analysis, where similar levels of cleaved PARP were observed between the control and p53 knock-down cells at the same cisplatin concentrations (FIG. 17).

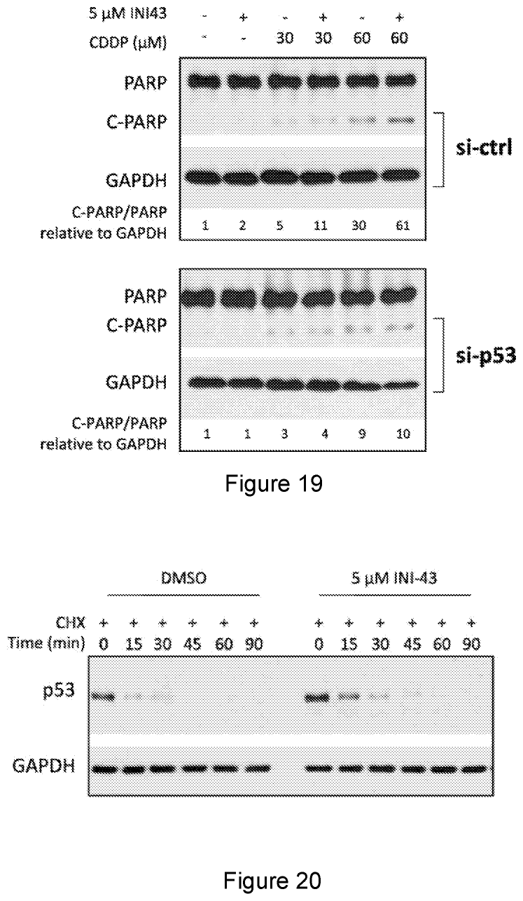

The impact of p53 on the enhancement of cell death observed after INI-43 and cisplatin CT was next examined. To quantify the “additional” cell death associated with the CT, cell viability was normalized to single cisplatin treatment. As previously established, a significant reduction in cell viability was observed after INI-43 and cisplatin CT, compared to single cisplatin treatment in the si-ctrl transfected cells. However, p53 knock-down cells exhibited similar sensitivity to single and CT, i.e., INI-43 pre-treatment induced sensitisation to cisplatin was lost with p53 inhibition (FIG. 18). Examination of PARP cleavage in these cells showed similar results; while si-ctrl transfected cells showed increased levels of cleaved PARP after CT compared to single cisplatin treatment, p53 knock-down cells exhibited similar levels of cleaved PARP between single and CT (FIG. 19). These results showed that cisplatin alone induced cell death is p53-independent, however, p53 appears to be critical for the enhancement of cell death observed in the CT, as p53 knock-down abrogated this effect.

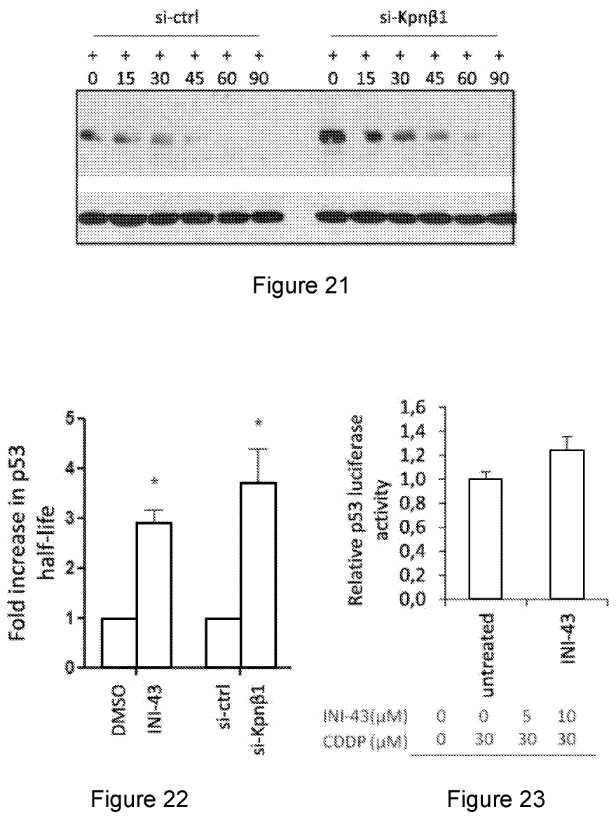

INI-43 Pre-Treatment Stabilized p53 Via Kpnβ1 Inhibition p53 is known to be highly unstable in HPV positive cells due to the activity of HPV oncoprotein E6 (Crook T, Tidy J A, Vousden K H. Degradation of p53 can be targeted by HPV E6 sequences distinct from those required for p53 binding and trans-activation. Cell. 1991; 67(3):547-56), and as SiHa is an HPV 16 positive cell line known to express E6 (Meissner J D. Nucleotide sequences and further characterization of human papillomavirus DNA present in the CaSki, SiHa and HeLa cervical carcinoma cell lines. J Gen Virol. 1999;80(Pt 7):1725-33), it was possible that INI-43 treatment interfered with p53 stability, thereby altering cell sensitivity to cisplatin treatment. To test this, the rate of p53 degradation was monitored in cyclohexmide (CHX)-treated cells. Cells were treated with 5 μM INI-43 or DMSO for 2 h, whereafter CHX was added and protein extracted at various time points after CHX treatment. Western blot analysis showed an increase in p53 stability in INI-43 treated cells compared to DMSO treated control cells (FIG. 20). To confirm that the prolonged p53 presence observed after INI-43 treatment was associated with Kpnβ1 inhibition, p53 levels were also examined in Kpnβ1 knock-down cells after CHX treatment. Similar to that observed after INI-43 treatment, Kpnβ1 knock-down cells were able to sustain p53 for a longer period after CHX treatment (FIG. 21). The half-life of p53 was calculated and an approximate 2.9-fold and 3.7-fold increase in half-life was observed, in INI-43 treated and si-Kpnβ1 transfected cells, respectively, compared to control cells (FIG. 22). Similar observations were made in HeLa cells, where Kpnβ1 knock-down increased p53 half-life by approximately 3.3-fold (data not shown). To investigate whether the stabilization of p53 had any functional relevance, p53 reporter activity was measured after INI-43 treatment. 5 μM INI-43 treatment led to a small but significant increase in p53 activity, consistent with its prolonged half-life (FIG. 23).

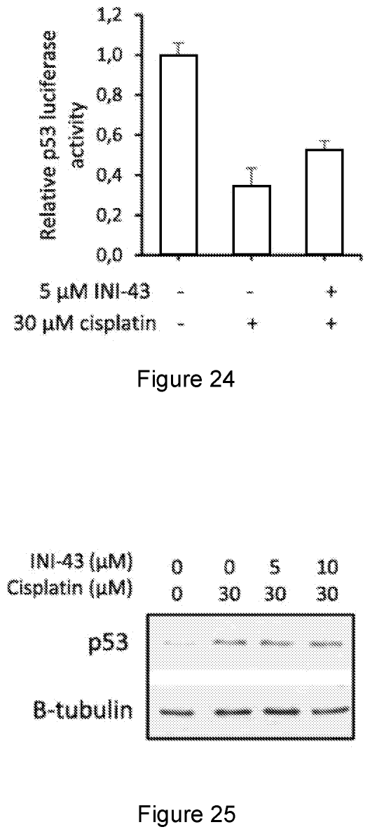

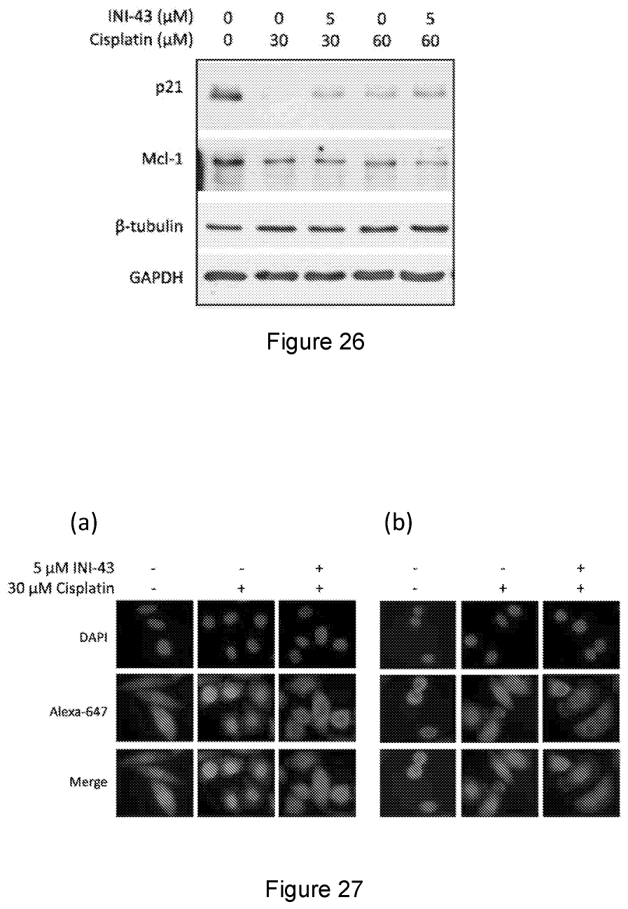

To relate these findings to combination treated cells, p53 reporter activity was measured in SiHa cells treated with INI-43 and cisplatin, compared to single cisplatin treatment. Interestingly, p53 reporter activity was significantly reduced upon single cisplatin treatment, in line with the lack of involvement of p53 in cisplatin-induced cell death observed in FIGS. 11 to 15 (FIG. 24). However, with INI-43 pre-treatment, p53 reporter activity was significantly increased (FIG. 24). p53 protein levels, on the other hand, were increased after single cisplatin treatment and after treatment with INI-43 and cisplatin (FIG. 25). These results show that while p53 is stabilized following treatment with cisplatin, its activity is inhibited. Pre-treatment with INI-43, however, results in increased p53 activity. Following from the increased p53 reporter activity, the levels of two proteins known to be regulated by p53 were investigated: p21 which is positively regulated by p53, and Mcl-1 which is transcriptionally repressed by p53. Western blot analysis showed that cisplatin treatment at 30 μM and 60 μM decreased levels of both p21 and Mcl-1. However, in cells receiving both INI-43 and cisplatin, p21 levels were elevated compared to single cisplatin treatment at both 30 and 60 μM concentrations, and Mcl-1 levels were reduced at 60 μM cisplatin (FIG. 26). These results confirm the involvement of p53 and p53 downstream targets in the INI-43-mediated enhanced cytotoxicity to cisplatin.

### INI-43-Cisplatin Combination Treatment Reduced Cisplatin-Induced Nuclear Accumulation of NFκB

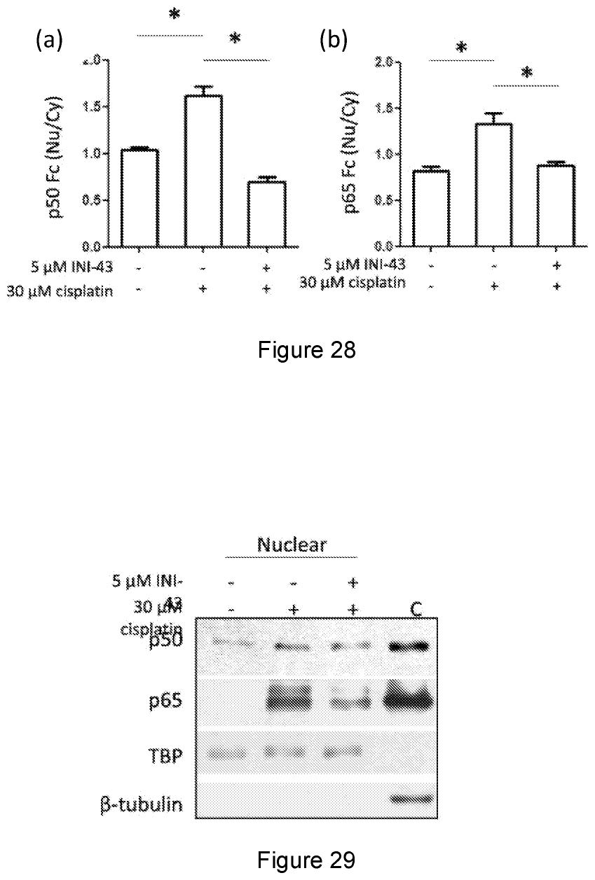

It has been previously shown that treating cancer cells with INI-43 prohibited PMA-stimulated nuclear entry of NFκB-p65 (van der Watt P J, Chi A, Stelma T, Stowell C, Strydom E, Carden S, et al. Targeting the nuclear import receptor Kpnbeta1 as an anticancer therapeutic. Mol Cancer Ther. 2016; 15(4):560-73). Others have reported that in SiHa cells, cisplatin treatment leads to activation of NFκB which contributes to cisplatin resistance in various cancer models (Godwin P, Baird A M, Heavey S, Barr M P, O'Byrne K J, Gately K. Targeting nuclear factor-kappa B to overcome resistance to chemotherapy. Front Oncol. 2013; 3:120). As NFκB activation requires nuclear translocation to initiate transcription of downstream targets, NFκB nuclear localization was evaluated by immunofluorescence after single and CT, as an indication of activity. Results showed that while cisplatin treatment stimulated nuclear localization of NFκB-p50 and NFκB-p65, INI-43 pre-treatment prevented this nuclear translocation of both NFκB subunits upon cisplatin treatment (FIG. 27). Fluorescence quantification supported these results, where cisplatin treatment led to a significant increase in nuclear fluorescence relative to cytoplasmic fluorescence (Fc (Nu/Cy)), and the INI-43-cisplatin CT significantly reduced this effect (FIG. 28).

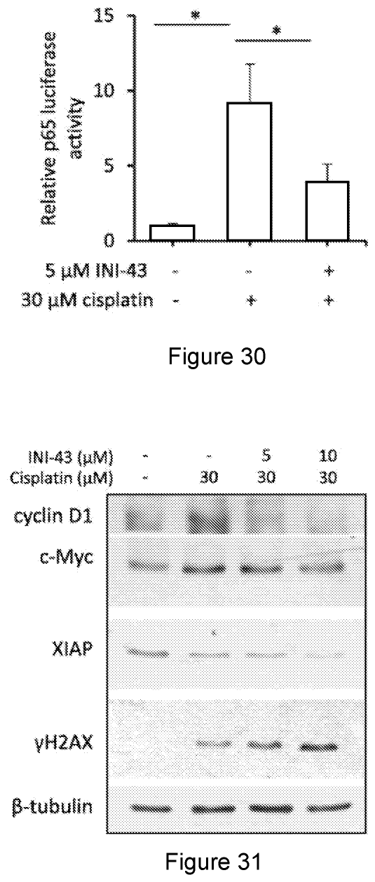

To independently confirm these results, nuclear and cytoplasmic protein fractions were isolated from cisplatin-treated or combination treated SiHa cells. Western blot analysis showed that cisplatin treatment resulted in increased levels of both NFκB-p50 and NFκB-p65 in the nucleus, and that INI-43-cisplatin CT reduced this effect (FIG. 29). Next, it was determined whether the altered localization of NFκB-p50 and NFκB-p65 after CT translated into functional significance. p65 reporter activity was measured after single or CT, and results showed that p65 reporter activity was induced upon cisplatin treatment, but the increase in p65 activity was reduced when cells were pre-treated with INI-43 (FIG. 30). The expression of three downstream targets of NFκB known to respond to cisplatin treatment were hence examined, namely cyclin D1, c-Myc and X Chromosome Linked Inhibitor of Apoptosis (XIAP) (Basu A, Krishnamurthy S. Cellular responses to Cisplatin-induced DNA damage. J Nucleic Acids. 2010; 2010:201367; Zhou X, Zhang Z, Yang X, Chen W, Zhang P. Inhibition of cyclin D1 expression by cyclin D1 shRNAs in human oral squamous cell carcinoma cells is associated with increased cisplatin chemosensitivity. Int J Cancer. 2009; 124(2):483-9; Xu B, Liu P, Li J, Lu H. C-MYC depletion potentiates cisplatin-induced apoptosis in head and neck squamous cell carcinoma: involvement of TSP-1 up-regulation. Ann Oncol. 2010; 21(3):670-2). Western blot analysis showed that single cisplatin treatment led to elevated levels of cyclin D1 and c-Myc (FIG. 31). Moreover, the levels of cyclin D1, c-Myc and XIAP were all reduced in INI-43-cisplatin combination treated cells in a concentration dependent manner compared to single cisplatin treated cells (FIG. 31). As both cyclin D1 and c-Myc have been shown to confer chemoresistance via increasing the cells' DNA repair capacity (Jirawatnotai S, Hu Y, Livingston D M, Sicinski P. Proteomic identification of a direct role for cyclin d1 in DNA damage repair. Cancer Res. 2012; 72(17):4289-93; Pyndiah S, Tanida S, Ahmed K M, Cassimere E K, Choe C, Sakamuro D. c-MYC suppresses BIN1 to release poly (ADP-ribose) polymerase 1: a mechanism by which cancer cells acquire cisplatin resistance. Sci Signal. 2011;4(166):ra19), it was examined whether their decreased levels observed in the CT had an impact on cisplatin-induced DNA damage. The level of phosphorylated Histone 2AX (γH2AX), a marker for DNA damage was examined 24 h after single or CT by western blot. Results showed that the INI-43-cisplatin CT increased γH2AX levels in a concentration dependent manner, suggesting that pre-treating cells with INI-43 prior to cisplatin treatment enhanced the DNA damaging effect of cisplatin (FIG. 31).

Together, these results demonstrate that INI-43 pre-treatment effectively reduced nuclear accumulation and activity of NFκB, resulting in decreased expression of cyclin D1, c-Myc and XIAP, and impaired DNA repair ability, sensitising the cells to cisplatin treatment.

Accordingly, the results demonstrate that inhibition of Kpnβ1 with a small molecule is an effective way to enhance the anti-cancer effects of cisplatin, and that both cisplatin sensitive, HeLa, and the more resistant, SiHa cervical cancer cells are responsive to this treatment. Furthermore, combination index analysis indicated a synergistic interaction between INI-43 and cisplatin, where their combined use produced greater anti-cancer effects compared to the added effects when used alone.

To understand the mechanism of action underlying the increased cisplatin sensitivity observed in the CT, proteins involved in cisplatin response were investigated, including both p53 and NFκB. Whilst p53 is widely accepted as a tumour suppressor protein important in guarding the genome and regulating apoptosis, some evidence has emerged to demonstrate that p53 can also promote oncogenesis by preventing apoptosis (Janicke R U, Sohn D, Schulze-Osthoff K. The dark side of a tumor suppressor: anti-apoptotic p53. Cell Death Differ. 2008; 15(6):959-76), suggesting that p53 can be involved in cisplatin resistance or cisplatin-induced apoptosis. P53 knock-down experiments demonstrated that p53 is involved in the pro-apoptotic pathway in the model system, but only in response to the CT, as p53 knock-down did not affect sensitivity to single cisplatin treatment.

SiHa cells are HPV positive, harbouring the HPV16 E6 oncoprotein (Meissner J D), which has been reported to directly associate with p53 and induce its degradation (Crook et al.). This results in a highly unstable p53, which is supported by the observation whereby p53 is rapidly degraded after CHX treatment. Stabilisation of p53 in response to INI-43 treatment and Kpnβ1 knock-down in SiHa cells has been previously observed. Stabilization of p53 has also been observed in HPV16 and HPV18 positive Kpnβ1 knock-down CaSki cells (Angus L, van der Watt P J, Leaner V D. Inhibition of the nuclear transporter, Kpnbeta1, results in prolonged mitotic arrest and activation of the intrinsic apoptotic pathway in cervical cancer cells. Carcinogenesis. 2014; 35(5):1121-31). The stabilisation of p53 upon Kpnβ1 inhibition is likely due to the role of Kpnβ1 in mediating p53 and HPV E6 nuclear entry. p53 is reported to enter the nucleus via Kpnα4 (Importin a3) and Kpnβ1 (Marchenko N D, Hanel W, Li D, Becker K, Reich N, Moll U M. Stress-mediated nuclear stabilization of p53 is regulated by ubiquitination and importin-alpha3 binding. Cell Death Differ. 2010; 17(2):255-67; Li O, Falsey R R, Gaitonde S, Sotello V, Kislin K, Martinez J D. Genetic analysis of p53 nuclear importation. Oncogene. 2007; 26(57):7885-93), however, it is known that there is redundancy between nuclear transport receptors, and it has previously been shown an accumulation of p53 in the nucleus and cytoplasm upon Kpnβ1 inhibition (Angus et al.), suggesting p53 still has access to the nucleus when Kpnβ1 is inhibited. HPV E6 protein has also been reported to enter the nucleus via Kpnβ1 and Kpnβ2 (Le Roux L G, Moroianu J. Nuclear entry of high-risk human papillomavirus type 16 E6 oncoprotein occurs via several pathways. J Virol. 2003; 77(4):2330-7). It is possible that Kpnβ1 inhibition with INI-43 affects nuclear entry of p53 and HPV E6 to varying extents, interfering with HPV E6-mediated p53 degradation, and resulting in p53 stabilisation. The exact mechanism involved, however, requires further investigation. Interestingly, inhibition of CRM1 via small molecules KPT-185 and leptomycin B has also been shown to stabilize p53 in other cancers (Wang S, Han X, Wang J, Yao J, Shi Y. Antitumor effects of a novel chromosome region maintenance 1 (CRM1) inhibitor on non-small cell lung cancer cells in vitro and in mouse tumor xenografts. PLoS One. 2014;9(3):e89848; Lecane P S, Kiviharju T M, Sellers R G, Peehl D M. Leptomycin B stabilizes and activates p53 in primary prostatic epithelial cells and induces apoptosis in the LNCaP cell line. Prostate. 2003; 54(4):258-67). Together with the results described herein, the data suggest that interfering with the nuclear transport system in either directions has stabilizing effects on p53.

In combination treated cells, there was increased p53 activity after INI-43 pre-treatment, which associated with increased responsiveness to cisplatin treatment. It is proposed that in the model system, p53 protein accumulates upon cisplatin treatment, however, the action of HPV E6 renders it inactive (Lechner M S, Laimins L A. Inhibition of p53 DNA binding by human papillomavirus E6 proteins. J Virol. 1994; 68(7):4262-73). p53 knock-down thus had little effect on cisplatin induced cell death. However, in the combination treated cells it is possible that the inhibition of Kpnβ1 interferes with p53 and HPV E6 nuclear entry, altering the levels of E6-bound p53 in the nucleus, and the p53 that accumulates is more readily available for apoptotic induction when cells are challenged with cisplatin. This may also explain why INI-43 did not sensitize C33A cells to cisplatin, as C33A cells are HPV negative and carry a non-functional mutant p53 (Crook T, Wrede D, Vousden K H. p53 point mutation in HPV negative human cervical carcinoma cell lines. Oncogene. 1991; 6(5):873-5).

In addition to enhanced p53 stability and reporter activity, increased p21 levels and decreased Mcl-1 levels were observed in INI-43 pre-treated cells compared to non-pre-treated cells in response to cisplatin treatment. p53 is known to positively regulate p21 expression and repress Mcl-1 (He G, Siddik Z H, Huang Z, Wang R, Koomen J, Kobayashi R, et al. Induction of p21 by p53 following DNA damage inhibits both Cdk4 and Cdk2 activities. Oncogene. 2005; 24(18):2929-43; Pietrzak M, Puzianowska-Kuznicka M. p53-dependent repression of the human MCL-1 gene encoding an anti-apoptotic member of the BCL-2 family: the role of Sp1 and of basic transcription factor binding sites in the MCL-1 promoter. Biol Chem. 2008; 389(4):383-93). Furthermore, the elevated caspase-3/7 activity observed in the CT could be associated with the decreased levels of Mcl-1, as Mcl-1 is known to promote survival by inhibiting events preceding mitochondrial release of cytochrome C (Clohessy J G, Zhuang J, de Boer J, Gil-Gomez G, Brady H J. Mcl-1 interacts with truncated bid and inhibits its induction of cytochrome c release and its role in receptor-mediated apoptosis. J Biol Chem. 2006; 281(9):5750-9).

Interestingly, with opposing roles in apoptosis, NFκB and p53 have been shown to mutually antagonize the transcriptional activity of each other (Webster G A, Perkins N D. Transcriptional cross talk between NF-kappaB and p53. Mol Cell Biol. 1999; 19(5):3485-95), and the results showed there was also a differential distribution of NFκB subunits p50 and p65 in cells receiving the single cisplatin and CT. NFκB is an important response factor to stress signals, including cisplatin-induced DNA damage (Volcic M, Karl S, Baumann B, Salles D, Daniel P, Fulda S, et al. NF-kappaB regulates DNA double-strand break repair in conjunction with BRCA1-CtlP complexes. Nucleic Acids Res. 2012; 40(1):181-95), whereupon it relocates to the nucleus to promote the transcription of various genes involved in DNA repair and survival (Godwin P et al.). As NFκB is reliant on Kpnβ1/Karyopherinα for nuclear entry (Yan W, Li R, He J, Du J, Hou J. Importin beta1 mediates nuclear factor-kappaB signal transduction into the nuclei of myeloma cells and affects their proliferation and apoptosis. Cell Signal. 2015; 27(4):851-9), the localisation of NFκB was measured after INI-43 treatment which showed that INI-43 inhibited cisplatin-induced nuclear import of NFκB, as well as the expression of its transcriptional targets cyclin D1, c-Myc and XIAP. This coincided with elevated levels of γH2AX, suggesting that Kpnβ1 inhibition either augmented the DNA damaging capacity of cisplatin, or, alternatively, impaired the DNA repair response. c-Myc confers chemoresistance via suppressing BIN1, an inhibitor of PARP-1 involved in DNA repair activity, thereby increasing tolerance to DNA damage and conferring cisplatin resistance (Pyndiah et al.). XIAP promotes survival by directly binding to and inhibiting the activities of caspase-3, caspase-7 and possibly caspase-9 (Salvesen G S, Duckett C S. IAP proteins: blocking the road to death's door. Nat Rev Mol Cell Biol. 2002; 3(6):401-10). Cyclin D1, best known for driving cell cycle from G1 to S phase, is also involved in DNA damage repair in association with Rad51 (Jirawatnotai S, Hu Y, Michowski W, Elias J E, Becks L, Bienvenu F, et al. A function for cyclin D1 in DNA repair uncovered by protein interactome analyses in human cancers. Nature. 2011; 474(7350):230-4), and its inhibition impairs DNA repair capacity leading to sensitization of cancer cells to cisplatin (Jirawatnotai et al.). The results show that INI-43-cisplatin CT results in reduced levels of these DNA-repair and anti-apoptotic proteins, possibly via decreasing NFκB nuclear import and transcriptional activity. However, the response of these proteins to INI-43-cisplatin CT may also be attributed to other mechanisms besides NFκB. For example, it has been shown that in addition to blocking NFκB nuclear translocation, Kpnβ1 inhibition also reduced the nuclear translocation of c-Myc in prostate cancer cells (Yang J, Guo Y, Lu C, Zhang R, Wang Y, Luo L, et al. Inhibition of Karyopherin beta 1 suppresses prostate cancer growth. Oncogene. 2019; 38(24):4700-14).

It has also been found that Kpnβ1 overexpression similarly sensitizes cervical cancer cells to cisplatin. However, overexpression of Kpnβ1 (above what is already expressed in the cancer cells) does not benefit cancer cell survival. Rather, it reduced cancerous properties including reduced cell proliferation, increased cell adhesion and mesenchymal-to-epithelial transition (Carden S, van der Watt P, Chi A, Ajayi-Smith A, Hadley K, Leaner V D. A tight balance of Karyopherin 61 expression is required in cervical cancer cells. BMC Cancer. 2018;18(1):1123). Hence, it appears that it is a tightly controlled balance of Kpnβ1 level that is beneficial to the cancerous traits, and that perturbation of this equilibrium in either direction (overexpression or inhibition) is detrimental to the survivability of cancer cells. Nevertheless, inhibition of Kpnβ1 is a more viable strategy and has more potential for therapeutic therapy in combination with cisplatin.

The results demonstrate that Kpnβ1 inhibition sensitizes cervical cancer cells to cisplatin, and that coupling nuclear import inhibition with platinum-based chemotherapeutic agents such as cisplatin may be an effective anti-cancer approach. This is believed to be mediated through stabilisation of p53 and prevention of NKκB nuclear localization, leading to alterations in the expression of various downstream targets such as XIAP, c-Myc, and Mcl-1. These proteins are known to confer cisplatin resistance in a variety of cancers, and their inhibition through genetic or pharmacological approaches have been demonstrated to increase sensitivity to chemotherapeutic agents (Xu B et al.; Dean E J, Ward T, Pinilla C, Houghten R, Welsh K, Makin G, et al. A small molecule inhibitor of XIAP induces apoptosis and synergises with vinorelbine and cisplatin in NSCLC. Br J Cancer. 2010; 102(1):97-103; You L, Wang Y, Jin Y, Qian W. Downregulation of mci-1 synergizes the apoptotic response to combined treatment with cisplatin and a novel fiber chimeric oncolytic adenovirus. Oncol Rep. 2012; 27(4):971-8). The abrogation of enhanced cell death in combination treated cells via p53 knock-down suggest that p53 is likely upstream of the NFκB-induced survival response.

The combination therapy described herein is particularly advantageous, since not only the anti-cancerous effect is enhanced compared to the effect of each compound/agent alone, the dosage of each agent in a combination therapy can be reduced as compared to monotherapy with each agent, while still achieving an overall anti-cancerous effect. In addition, due to the enhanced and/or synergistic effect, the total amount of drugs administered to a patient can be reduced, which may result in minimizing undesired side effects.

Furthermore, specific patients or specific types of cancers exhibiting moderate resistance to platinum-based chemotherapeutic agents may show an increased treatment response with the use of the combinations described herein. Alternatively, the same treatment outcome may be achieved with lower doses of the platinum-based chemotherapeutic agent to at least reduce its side-effects. In contrast to platinum-based drugs, the compounds of Formula I provide a more targeted anti-cancer effect as Kpnβ1 inhibition showed no toxicity on non-cancer cells.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. A combination for use in treating cancer comprising
(a) a therapeutically effective amount of a compound of Formula I: 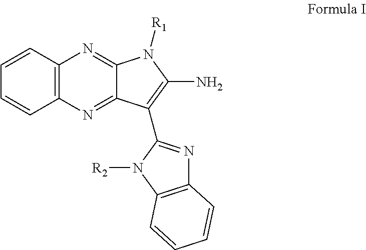
or a pharmaceutically acceptable salt thereof, wherein
R₁ is a branched or linear C2-C5 alkyl group optionally functionalised with a substituent selected from the group consisting of an amine, an imidazole, an alcohol or a morpholine; and
R₂ is a hydrogen or a methyl group; and
(b) a therapeutically effective amount of at least one platinum-based chemotherapeutic agent.

2. The combination as claimed in claim 1, wherein R₁ is selected from: 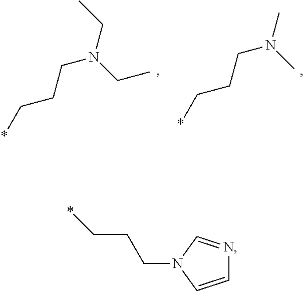
an ethyl group, a propyl group, a butyl group, an iso-butyl group, an iso-pentyl group, a propanol group and

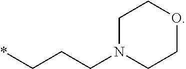 [SMILES: *CCCN1CCOCC1]

3. The combination as claimed in claim 1, wherein the compound of Formula I is selected from:

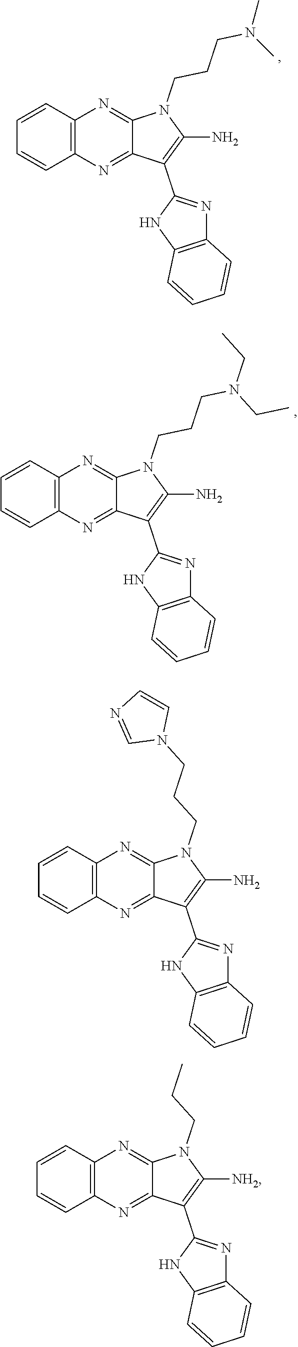

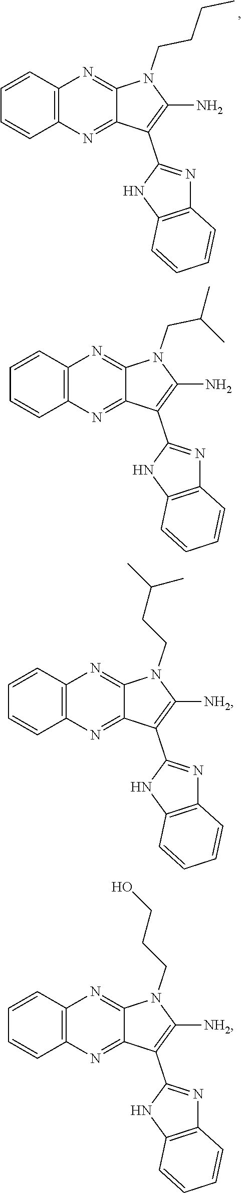

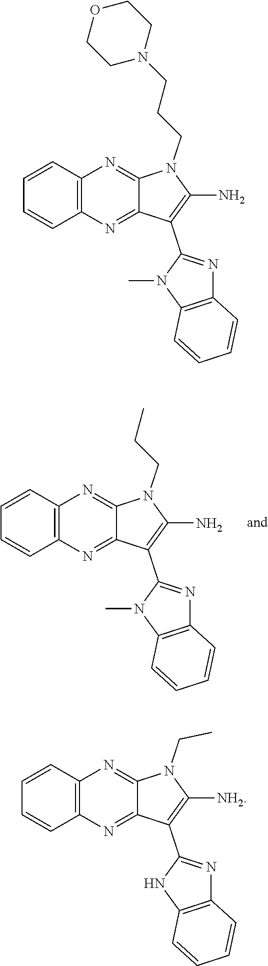 [SMILES: Cn1c(-c2c(N)n(CCCN3CCOCC3)c3nc4ccccc4nc23)nc2ccccc21 ; CCCn1c(N)c(-c2nc3ccccc3n2C)c2nc3ccccc3nc21 ; CCn1c(N)c(-c2nc3ccccc3[nH]2)c2nc3ccccc3nc21]

4. The combination as claimed in claim 1, wherein the compound of Formula I is

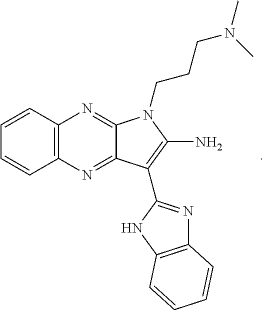 [SMILES: CN(C)CCCn1c(N)c(-c2nc3ccccc3[nH]2)c2nc3ccccc3nc21]

5. The combination as claimed in claim 1, wherein the combination provides an enhanced therapeutic anti-cancer effect.

6. The combination as claimed in claim 1, wherein the combination provides a synergistic therapeutic anti-cancer effect.

7. The combination as claimed in claim 1, wherein the cancer is selected from the group consisting of cervical cancer, oesophageal cancer, ovarian cancer, breast cancer and uterine cancer.

8. The combination as claimed in claim 1, wherein the at least one platinum-based chemotherapeutic agent is known to be effective in treating the cancer.

9. The combination as claimed in claim 1, wherein the platinum-based chemotherapeutic agent is selected from the group consisting of cisplatin, carboplatin, oxaliplatin, nedaplatin, lobaplatin and heptaplatin.

10. The combination as claimed in claim 9, wherein the platinum-based chemotherapeutic agent is cisplatin.

11. A method for treating cancer comprising administering to a subject in need thereof a compound of Formula I: 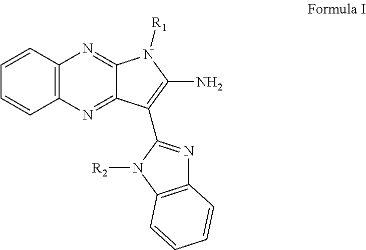
or a pharmaceutically acceptable salt thereof, wherein
R₁ is a C2-C5 alkyl group, branched or linear, optionally functionalised with a substituent selected from the group consisting of an amine, an imidazole, an alcohol or a morpholine; and
R₂ is a hydrogen or a methyl group, in combination with at least one platinum-based chemotherapeutic agent.

12. The method of claim 11, wherein the method results in an enhanced therapeutic effect compared to the effect of the compound of Formula I and the at least one platinum-based chemotherapeutic agent administered alone.

13. (canceled)

14. The method of claim 11, wherein the compound of Formula I is administered simultaneously with the at least one platinum-based chemotherapeutic agent.

15. The method of claim 11, wherein the compound of Formula I and the at least one platinum-based chemotherapeutic agent are administered sequentially.

16. A kit for treating cancer comprising
(a) a therapeutically effective amount of a compound of Formula I: 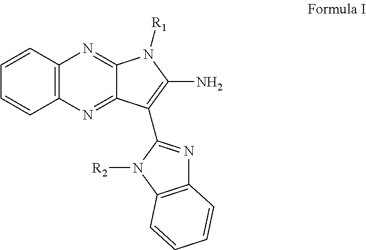
or a pharmaceutically acceptable salt thereof, wherein
R₁ is a branched or linear C2-C5 alkyl group optionally functionalised with a substituent selected from the group consisting of an amine, an imidazole, an alcohol or a morpholine; and
R₂ is a hydrogen or a methyl group; and
(b) a therapeutically effective amount of at least one platinum-based chemotherapeutic agent.
